(19) United States
(12) Patent Application Publication
(10) Pub. No.: US 2011/0212453 A1
(43) Pub. Date: Sep. 1, 2011

(54) ASSAY CARD FOR SAMPLE ACQUISITION, TREATMENT AND REACTION

(76) Inventors: Abhishek K. Agarwal, Evanston, IL (US); Sujit R. Jangam, San Mateo, CA (US); Kunal Sur, Chicago, IL (US); David M. Kelso, Wilmette, IL (US)

(21) Appl. No.: 13/027,147

(22) Filed: Feb. 14, 2011

Related U.S. Application Data
(60) Provisional application No. 61/304,018, filed on Feb. 12, 2010.

(51) Int. Cl. C12Q 1/68; C12M 1/34; G01N 21/00; B01L 3/00

(52) U.S. Cl. 435/6.12; 435/287.2; 422/82.05; 422/501

(57) ABSTRACT

The present disclosure relates to devices and systems and methods for their use for detecting an analyte. In particular, the present disclosure provides a disposable assay card in which reaction reagents are stored within the card to facilitate point-of-care application.

### CROSS-REFERENCE TO RELATED APPLICATION

This application claims priority to U.S. Provisional Application Ser. No. 61/304,018 filed Feb. 12, 2010, which is herein incorporated by reference in its entirety.

### TECHNICAL FIELD

The present disclosure relates generally to the field of analyte detection, and more specifically to an assay device that receives and processes a sample for detection of an analyte. Component parts of the assay device, systems comprising the assay device and its component parts, and methods of use are also described.

### BACKGROUND

Determining the presence or absence of a particular analyte in biological and environmental samples is desired for a variety of diagnostic, forensic and monitoring purposes. There is a particular need in the art for devices capable of analysis of a sample at a point-of-care site or laboratory, or out in the field, where sample preparation, reagent introduction and transfer, and detection steps need to be minimized and simplified. The present reaction card serves this need.

### BRIEF SUMMARY

In one aspect, an assay device is provided. The assay device comprises a planar reaction card comprising a sample port for receiving a sample into a reaction chamber, and a reagent input port on the reaction card for receiving a reagent, the reagent input port in fluid communication with the reaction chamber. The assay device also comprises at least one reagent reservoir in fluid communication with the reagent input port and attached to the card, and an optical window positioned on the card for monitoring a reaction in the reaction chamber.

In one embodiment, the reaction chamber is directly adjacent the sample port such that upon introduction of a sample via the sample port, the sample is in the reaction chamber.

In another embodiment, the at least one reagent reservoir comprises a storage chamber comprising a reagent in liquid form. An elongate collar encloses the storage chamber and serves as an area by which the reagent reservoir can be affixed to the reaction card.

In another embodiment, the assay device further comprises an attachment member for securing the reagent reservoir to a face of the reaction card, the attachment member having at least one opening that aligns with the reagent input port when the attachment member is secured to the reaction card.

In still another embodiment, a clamp that encloses the reagent reservoir is included on the device, the clamp having a wall with a height greater than a height of the storage chamber of the reaction reservoir.

In yet another embodiment, the at least one reagent reservoir comprises a reagent in dried form deposited in the reaction chamber or in a reagent input channel that connects the reagent input channel and the reaction chamber.

The device, in still another embodiment, comprises a second reagent reservoir attached to an external face of the reaction card by an attachment member, the attachment member having an opening that aligns with the reagent input port on the reaction card.

In one embodiment, a sample introduction module is configured to engage the sample port, the sample introduction module comprising a carrier on which a sample can be captured and a sealing means for engaging the sample introduction module with the sample port.

In another embodiment, the assay device further comprises an exit port in fluid communication with the reaction chamber. In one embodiment, a liquid impermeable, gas permeable membrane is positioned over the exit port. In still another embodiment, the membrane is a part of an attachment member that secures a reagent reservoir to the reaction card.

In yet another embodiment, the reagent reservoir is comprised of a frangible storage chamber capable of bursting open upon application of force.

In another embodiment, the reaction card has a selected thickness to define an edge with the selected thickness, and the optical window is disposed on the edge in a region adjacent the reaction chamber.

In another aspect, an assay card is provided. The assay card comprises a sample introduction module movably engaged with an opening on the assay card, the sample introduction module forming a liquid-tight seal with the opening when engaged therein. The sample introduction module is movable from a first position to at least one subsequent position in the assay card. One or more reagent reservoirs is/are positioned on the assay card for release of a component when the sample introduction module is in the first position or the at least one subsequent position.

In one embodiment, a sample is dispensed onto the sample introduction module when in the first position, the sample dispensed through a sample port in said assay card, the sample port in fluid communication with the sample introduction module when it is in the first position.

In another embodiment, the at least one subsequent position is a second position or a third position, and one of the second position and the third position corresponds to a reaction chamber.

In yet another embodiment, the reaction chamber comprises a reagent in dried form.

In still another embodiment, a reagent reservoir is positioned on the assay card to dispense a liquid reagent from the reagent reservoir through a reagent input port on the assay card that is in fluid communication with the reaction chamber.

In one embodiment, the at least one subsequent position comprises a second position and a third position, and a reagent reservoir is associated with the second position to dispense a liquid reagent onto the sample introduction module when positioned in the second position, and a reagent reservoir is associated with the third position to dispense a liquid reagent onto the sample introduction module when positioned in the third position.

In yet another aspect, a kit for analyzing a sample for the presence or absence of an analyte is provided. The kit includes a planar reaction card comprising a sample port for receiving a sample into a reaction chamber; a reagent input port on the card for receiving a reagent, the reagent input port in fluid communication with the reaction chamber, an optical window positioned on the card for monitoring a reaction between the sample and the reagent in the reaction chamber. The kit also includes one or more reagent reservoirs configured to be affixed to the reaction card for release of a liquid component contained in a storage chamber on each of the one or more reagent reservoirs into the reagent input port of the reaction card. The kit also includes a sample introduction module configured for insertion into the sample port on the reaction card.

In one embodiment of the kit, the one or more reagent reservoirs comprise a first reagent reservoir comprising a buffer and a second reagent reservoir comprising reagents for a polymerase chain reaction.

In another embodiment, the kit also includes an attachment member for securing a reagent reservoir in the one or more reagent reservoirs to the reaction card.

In still another embodiment, the sample introduction module comprises a carrier member comprising a lysing agent.

In another aspect, a system is provided. The system comprises an assay card as described above, and an analyzer adapted for receiving the assay card, the analyzer comprising a thermal cycler, an electromechanical component to apply a force to the at least one reagent reservoir to effect release of the liquid reagent, and an optical system.

In one embodiment, the optical system comprises a light source for transmitting light at excitation wavelengths to the reaction chamber and a detector for detecting light at emission wavelengths from the reaction chamber.

In another embodiment, the electromechanical component comprises a force sensor.

In yet another embodiment, the thermal cycler is in contact with the reaction chamber of the assay card by a thermally-conductive intervening member.

In still another aspect, a method for detecting the presence or absence of an analyte in a sample is provided. The method comprises providing an assay card as described herein, placing a sample on the assay card, conducting a reaction in the reaction chamber between the sample and the reagent; and monitoring the reaction optically via the optical window to detect the presence or absence of an analyte in the sample.

In one embodiment, the sample is a biological sample. In a specific embodiment, the sample is blood.

In another embodiment, the analyte is a nucleic acid. In a particular embodiment, the nucleic acid is a viral nucleic acid.

### BRIEF DESCRIPTION OF THE DRAWINGS

FIGS. 1A-1C are a top plan view and a top perspective view of the front side of a reaction card (FIGS. 1A-1B, respectively) and a top view of the back side of the reaction card (FIG. 10), in accord with one embodiment;

FIG. 2 shows a top view of a back of a reaction card, wherein a handle portion can optionally include an identifier tag;

FIGS. 3A-3B show an exploded view of a portion of the reaction card, comprising the reaction chamber and fluidic channels in communication with the reaction chamber (FIG. 3A) and a wall configured to mate with the portion of the reaction card shown by the dashed line to enclose the chamber and channels (FIG. 3B);

FIGS. 4A-4B show front (FIG. 4A) and back (FIG. 4B) sides of a reaction card where the planar base as fabricated lacks walls to define the reaction chamber, leaving a through-hole or open region in the base, wherein external walls can be attached to define the reaction chamber;

FIG. 5 shows a reaction card adjacent where an arrangement of light emitting diodes (LEDs) and photosensors in an analyzer externally positioned adjacent an optical window of the reaction card;

FIGS. 6A-6B are schematic representations of an embodiment of a sample introduction module;

FIGS. 7A-7E are schematic representations showing additional details of embodiments of sample introduction modules when viewed from different perspectives;

FIG. 8 is a schematic of another embodiment of a reaction card on which one or more reagents have been deposited, forming an assay card, also referred to as an assay device;

FIG. 9 is a schematic of an assay card comprised of a reaction card and a reagent reservoir affixed to one face of the reaction card;

FIGS. 10A-10B are illustrations of the reagent reservoir (FIG. 10A) and an attachment member (FIG. 10B) that mates with the reagent reservoir and the reaction card, to form an assay card;

FIG. 11 shows a reagent reservoir and an attachment member to secure the reagent reservoir to a reaction card;

FIGS. 12A-12B show another embodiment of an attachment member for securing a reagent reservoir to a reaction card;

FIGS. 13A-13B illustrate a reagent reservoir secured to a reaction card and protected by a clamp;

FIGS. 14A-14C show schematic illustrations of embodiments of a clamp that nests over a reagent reservoir and protects the burstabie chamber in which reagents are stored in the reagent reservoir;

FIGS. 15A-15C are illustrations of clamps of two embodiments integrated with a reagent reservoir;

FIGS. 16A-16B are illustrations of an assay card in side view (FIG. 16A) and in perspective view (FIG. 16B), the assay card comprised of a SIM, a reaction card, and a reagent;

FIG. 17 illustrates steps in accord with a method for using an assay card as described herein;

FIGS. 18A-18C show another embodiment of an assay card;

FIGS. 19A-19D show side-view schematics of another embodiment of an assay card and its use in sample processing and detection of an analyte;

FIGS. 20A-20B show embodiments of an analyzer designed to receive an assay card as described herein, where the analyzer includes a thermal cycler and a crusher to release reagent from a reagent reservoir;

FIG. 21 shows various components of an electromechanical fluid delivery (EFD) sub-system of an analyzer used in conjunction with the assay card;

FIG. 22 is a graph of force measured by a force sensor as a function of time, in milliseconds, the force corresponding to the force resulting during the blister crushing process in an instrument that interacts with an assay card;

FIG. 23 is a graph of the temperature, in ° C., as a function of time, in seconds, of left and right sleeves of an analyzer during thermal cycling of an assay card, as compared to the set point;

FIG. 24A is a graph of fluorescence (as an arbitrary fluorescent unit) as a function of cycle number, which correlates to the number of amplicons during RT-PCR of a blood sample placed on an assay card as described herein and analyzed for HIV-1; and

FIG. 24B shows the fluorescence data in the Quasar 670 channel after background subtraction.

### DETAILED DESCRIPTION

In one aspect, an assay device, also referred to as an assay card, for receiving a sample, processing the sample and conducting a reaction on the processed sample is provided. The assay card provides in a single reaction chamber of the card a sample-to-answer solution for determining whether an analyte in the sample is present or absent. As will be illustrated below, the assay card is configured to receive a sample placed by a user on a sample introduction module, and to process and react the sample with reagents integrated into the assay card, thus not requiring manipulation of the sample by a user and eliminating the need for a user to pipette or add reagents to the card after the sample is loaded.

The assay device is comprised of a reaction card that receives the sample, which in one embodiment is carried on a sample introduction module, and reagents, disposed within a channel or chamber of the reaction card or attached as an external reservoir reagent to a face of the reaction card. The sample is placed directly into the reaction chamber in the reaction card. Reagents for processing the sample and/or reacting with the sample are present on the reaction card or are introduced into the reaction chamber by means of one or more reagent reservoirs that mate with the reaction card in such a way as to dispense a reagent into the reaction card. Section A below describes the reaction card, Section B below describes the sample introduction module, and Section C below describes the reagent reservoirs. Section D then illustrates use of the assay device, composed of the reaction card, sample introduction module and reagent reservoirs for analysis of the presence or absence of an analyte in a sample.

### A. REACTION CARD

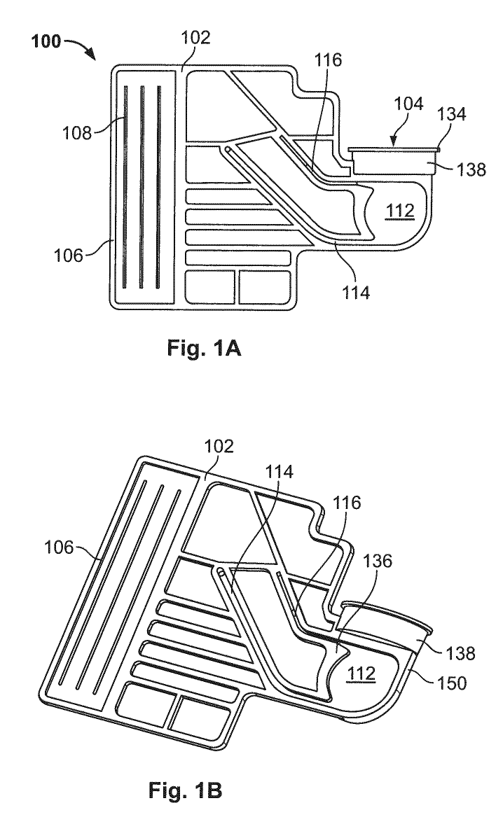

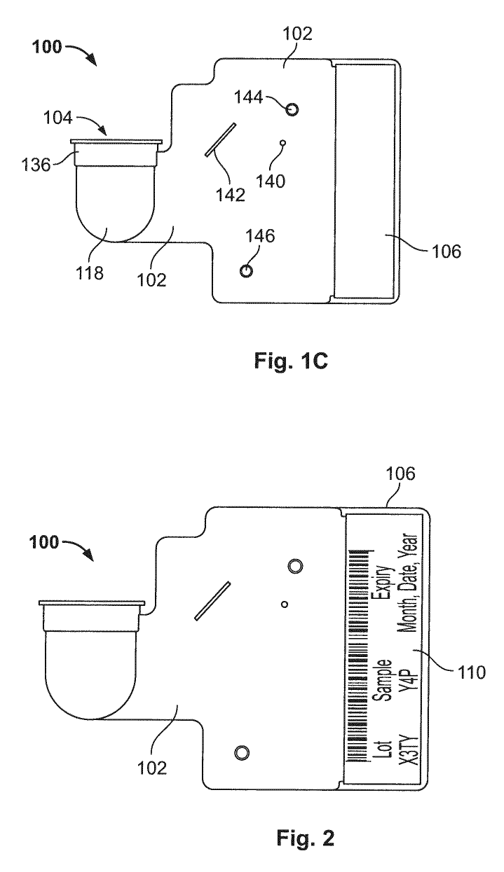

FIGS. 1A-1C show a first embodiment of a reaction card 100. With initial reference to FIGS. 1A-1B, which show a first, or front, view of the planar reaction card in top view (FIG. 1A) and in perspective (FIG. 1B), card 100 comprises a planar base 102. The planar base has a first and second external faces corresponding to the front side and back side of the reaction card. Planar base 102 includes a port 104 for introduction of a sample, and in a preferred embodiment, for introduction of a sample introduction module which is described herein below. As used herein, “planar” intends that the thickness of the planar base (or reaction card) is less than the smaller of the width or length of the planar base (or reaction card), where in one embodiment the thickness of the base or card is 2, 3, 4, or 5 times less than the width or the length of the base or card. The reaction card also comprises a handle portion 106, which can optionally be textured to secure handling by a user, and exemplary ribs, such as rib 108, are visible on the front view of the reaction card. Handle portion 106 can also optionally serve as an area to affix a label or identification tag to the reaction card, as illustrated in FIG. 2, where label 110 is positioned on the back of card 100 in portion 106. Label 110 can include any information useful to a user or potential user of the card, such as patient name, date of sample collection, assay lot number, expiration date of reagents pre-loaded on the card (if any, discussed herein below), sample number, etc. The label can additionally or optionally include machine-readable information, such as a bar code as shown or as a radio-frequency identifier (REID) to be read by an external instrument. The label, in other embodiments, serves to provide one or more combinations of the following information: (1) correlation of the sample or the sample introduction module (described below) with the reaction card by identifying the specific patient from whom the sample was collected, (2) assay lot number, (3) assay parameters, (4) expiration date of card or reagents on the card, and (5) type of assay to be performed.

With reference again to FIGS. 1A-1C, card 100 comprises a reaction chamber 112, visible best from the front, top view of the card as in FIGS. 1A-1B. Reaction chamber 112 is in communication with port 104 to receive a sample into the reaction chamber for processing, as described below. Reaction chamber 112 is in fluid communication with a reagent input channel 114 and an exit channel 116. Reaction chamber 112 is defined by a first wall (or floor, depending on orientation of the card), designated as 118 in FIG. 1C. In one embodiment, wall 118 is manufactured with a slight outward (positive) draft or bow, to define chamber 112 and to ensure thermal contact with an external thermal cycler instrument during use of the card.

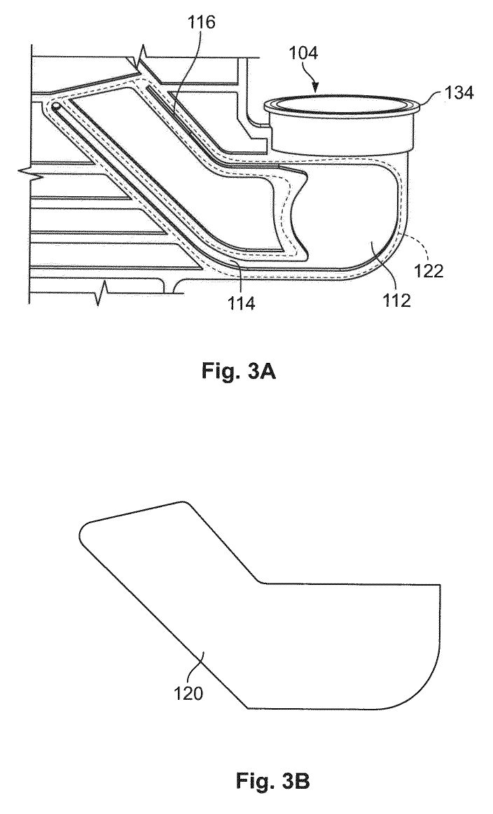

FIGS. 3A-3B show an exploded view of the reaction chamber 112, reagent input channel 114 and exit channel 116 of the reaction card (FIG. 3A) and a second wall 120 (or floor, depending on orientation of the card: FIG. 3B) configured to mate with the portion of the reaction card outlined by a dashed line 122. The first wall of reaction chamber 112 is generally formed during manufacture of the reaction card, for example, during a molding process of the planar reaction card. The first wall is preferably molded to have a wall thickness that maximizes thermal transfer efficiency yet provides sufficient structural integrity during handling and use, especially during thermal cycling. The second wall when mated with the reaction card encloses the reagent input channel, the exit channel, and the reaction chamber. The second wall is secured to the base of the reaction card is any one of a variety of suitable means, from affixing with an adhesive to bonding via ultrasonic or heat welding. In one embodiment, the base of the reaction card is molded to have a raised ledge to which the second wall is affixed. The second wall is secured to the planar base by a means that provides a vapor-tight and liquid-tight seal. Energy directors may be molded onto the planar base to facilitate ultrasonic welding, laser welding or heat welding of the second wall, when the wall is bonded by these methods.

The reaction chamber defined by the first and second walls is the primary site of chemical reaction on the card, where the sample is processed for analysis, as will be described. In the illustrated embodiments in the drawings, the reaction chamber is generally circular or spherical, however a skilled artisan will understand that the reaction chamber can have another geometry. The volume of the reaction chamber is determined at least in part by the assay specifications, and may be scaled accordingly for particular assay requirements. Reagents can also be coated or disposed in the reaction chamber. The walls and dimensions of the reaction chamber are preferably smooth with no sharp corners or edges to prevent air bubble entrapment. In a preferred embodiment, the reaction chamber has a surface-to volume-ratio of greater than 1, to maximize thermal transfer from an externally applied heat source. More preferably, the surface-to-volume ratio of the reaction chamber area, measured as the area of the walls enclosing the chamber, to the liquid volume in a reaction chamber during operation of the assay card is greater than 1, preferably greater than about 1.2, more preferably greater than about 1.5, about 1.8, about 2.0. In certain examples, the volume of the reaction chamber is between about 100-600 μL, preferably about 200-500 μL, more preferably about 200-400 μL, still more preferably about 200-300 μL. In another embodiment, the area of the walls of the reaction chamber is between about 100-500 mm², preferably about 150-400 mm², more preferably about 200-350 mm², still more preferably about 200-300 mm².

As can be appreciated from the foregoing description of planar base and the walls, the first and second walls defining the reaction chamber can both be rigid, can both be flexible, or can be configured so one is rigid and one is flexible. The material from which the walls are manufactured can and will vary, determined in part by selection of material(s) compatible with the sample, the reagents, and the assay conditions and protocol. An exemplary flexible material for use as the second wall is a thermally conductive polymers, such as the CoolPoly series of thermally conductive polymers (Cool Polymers, Inc. Warwick, R.I., USA). A benefit of a flexible material is that the wall can flex during use, to expand upon addition of reagent to the reaction chamber, thereby promoting good thermal contact with an external heat source, such as thermal cycler instrumentation. Alternatively, either wall can be a thicker, more rigid wall, formed of a thermally conductive polymer to a thickness (e.g. thickness>0.008 inch) that offers some structural rigidity by virtue of its thickness or from a rigid material.

In one embodiment, the second wall is a flexible wall, and is attached to the rigid planar base by a laser welding process, and a laser-absorbing dye is added to one or both of the materials forming the walls of the reaction chamber. The laser-absorbing dye can be integrated into the materials by adding it to the resin pellets during an injection molding process of the planar base of the reaction card. Alternatively, the dye can be applied to all or selected regions of the planar base of the reaction card after the injection molding process (before the laser welding) using a roller application, spray, needle tip dispensing, or hot stamping. If ultrasonic welding is used, the design of the ledge on the processor card must be modified to include energy directors to facilitate bonding of the film to the processor card.

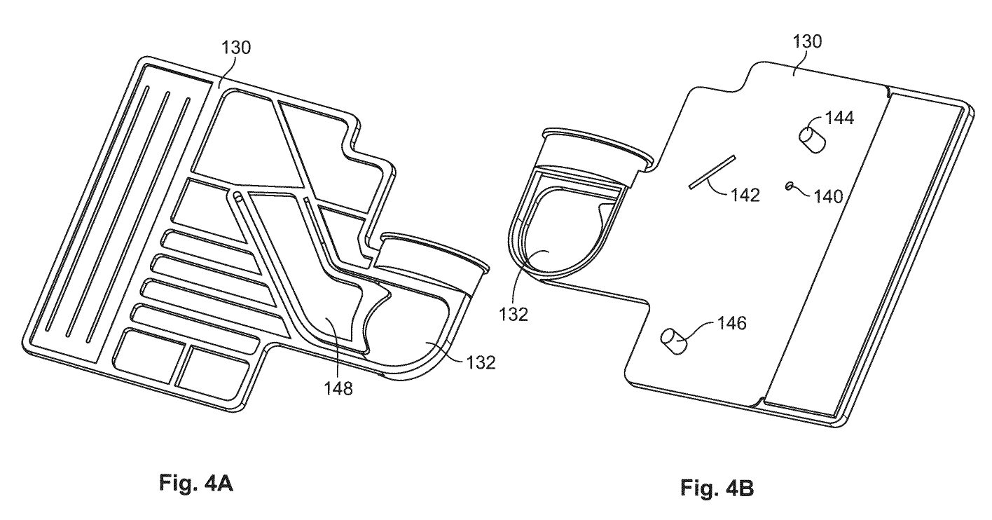

Another embodiment of a planar base for formation of a reaction card is shown in FIGS. 4A-4B. In this embodiment, planar base 130 as manufactured lacks walls to define a reaction chamber. As manufactured, a through-hole or open region 132 is defined by a gap in the planar base. This embodiment of a reaction card allows for attachment of first and second walls to define the reaction chamber, permitting selection of suitable wall materials for the reaction chamber, to, for example, optimize thermal transfer or to, ensure compatibility with the assay reactants and conditions. Alternatively, a rigid or flexible wall can be attached (as described above with reference to FIGS. 3A-3B) and a sample introduction module, to be described below, can define the second wall, so that the first wall and second wall together define the reaction chamber.

With reference again to FIGS. 1A-1C, port 104 is configured to receive a sample, and preferably a sample carried on a sample introduction module, described below. The port in the embodiment illustrated is circular or elliptical in shape, and in one embodiment is polarized such that a sample introduction module can be fully engaged with the card in a single orientation. A lip or flange 134 surrounds the port, and the sample introduction module when inserted into the port seats against the flange, creating a stop for insertion of the sample introduction module and creating a liquid-tight seal. An elliptical profile of the port offers a benefit in minimizing the overall size of the reaction card in the z direction, maximizing packaging capabilities in a given sized box. One or more guides can be included in the port, such as guide 136 and optionally other guides (not visible) in a neck region 138 of the port region to guide the sample introduction module during insertion, ensuring the sample introduction module is positioned correctly in the reaction card.

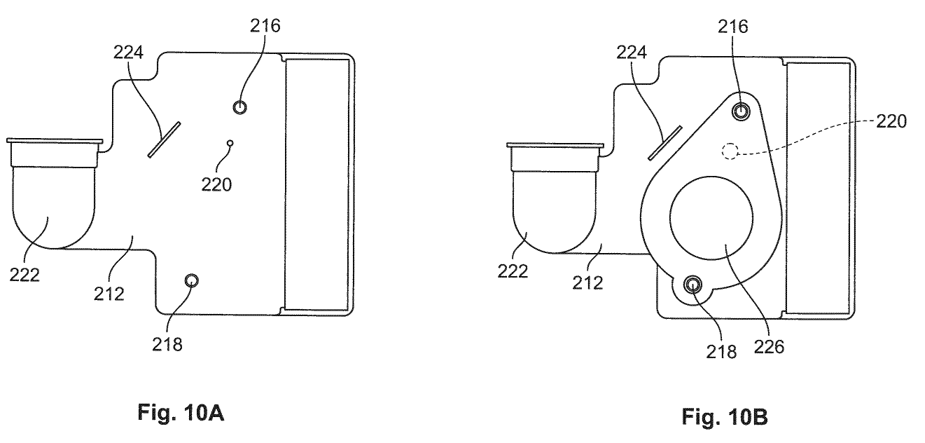

Reaction card 100 also comprises a reagent input port 140, visible in FIG. 10. The reagent input port is in fluid communication with reagent input channel 114 (FIG. 1A). In one embodiment, a reagent reservoir (described below) aligns with the input port on the reaction card to permit introduction of one or more reagents, generally in liquid form. The liquid then flows into the reagent input channel and into the reaction chamber. Freeze dried and/or gel stored reagent(s) can be positioned along or within the reagent input channel or in the reaction chamber, and reconstituted with a liquid from the sample or a liquid introduced via the reagent input port. The width, depth, and smooth contour geometry of the reagent input channel are designed to enable smooth laminar flow of the liquid reagent and prevent bubble entrapment.

The reaction card also comprises an exit port 142 (FIG. 10) in fluid communication with exit channel 116 (FIG. 1A). The exit port facilitates air venting from the reaction chamber when the sample or reagent is introduced into reaction chamber. Reagent will flow into the reaction chamber, displacing air, and will continue to flow into the exit channel, stopping at the exit port once all the air has been vented. If desired a gas permeable, liquid impermeable film or membrane can be placed over the exit port to ensure no liquid flows out of the exit port yet allows release of air from the chambers and channels of the reaction card as reagent flows into the card. The dimensions of the input channel, exit channel and reaction chamber and the amount of reagent introduced into the reaction card can each be selected so that in use the reaction card is pressurized by the liquid reagent. This causes the wall or walls, if flexible, of the reaction chamber to bulge outward, for good thermal contact with an external heat source. The exit channel's width, depth, and smooth contour promote smooth laminar flow of the reagent. The exit channel is also preferably disposed on the reaction card at the highest point to facilitate any air bubble movement to the top of the reaction card. Design of the reaction card so that when inserted into an external instrument, such as a thermal cycler, is at an upward tilt with the exit port at the highest position will also facilitate movement of any air bubbles in the channels or chambers of the reaction card toward the exit port.

As will be described below, the reaction card also comprises alignment pins or holes, such as pins 144, 146, seen best in FIG. 1C and FIG. 4B, as may be needed for alignment of the card with components parts or instrumentation for use.

The planar base of the reaction card can be manufactured to include one or more depressions or cavities, to reduce the material required for manufacture. The planar base can also be manufactured to include one or more ribs, the ribs serving to uniformly route the molten material (e.g., plastic) during fabrication, prevent warping of the card after the molding process, and/or to add structural integrity to the thin reaction card, which is beneficial for handling the card, either manually or inside an instrument.

In one embodiment, the reaction card includes a chamber, such as chamber 148 seen best in FIG. 4A, which is strategically positioned next to reaction chamber 132 to thermally isolate the reaction chamber as much as possible from the rest of reaction card. As will be described below, in use the reaction card is inserted into a thermal cycler to conduct an assay involving amplification of a nucleic acid in the sample by, for example, polymerase chain reaction. The open air chamber adjacent the reaction chamber promotes faster thermal cycling (i.e., less energy transfer away from the reaction chamber site to the rest of the reaction card.

The overall configuration of the reaction card can be varied. The configuration illustrated in FIGS. 1-4 is merely exemplary of the curves and edging that can be tailored for interaction of the card with a thermal cycler or other instrument for conducting the assay. The reaction card, in one embodiment, can comprise an internal surface finish to ensure smoothness and laminar flow. The reaction card, in certain embodiments, is injection molded from a plastic, such as polyethylene, polypropylene, polystyrene, or any of the thermoplastic materials typically used in the plastics molding industry. The material is chosen based on, for example, compatibility with the assay, cost, and ease of injection molding.

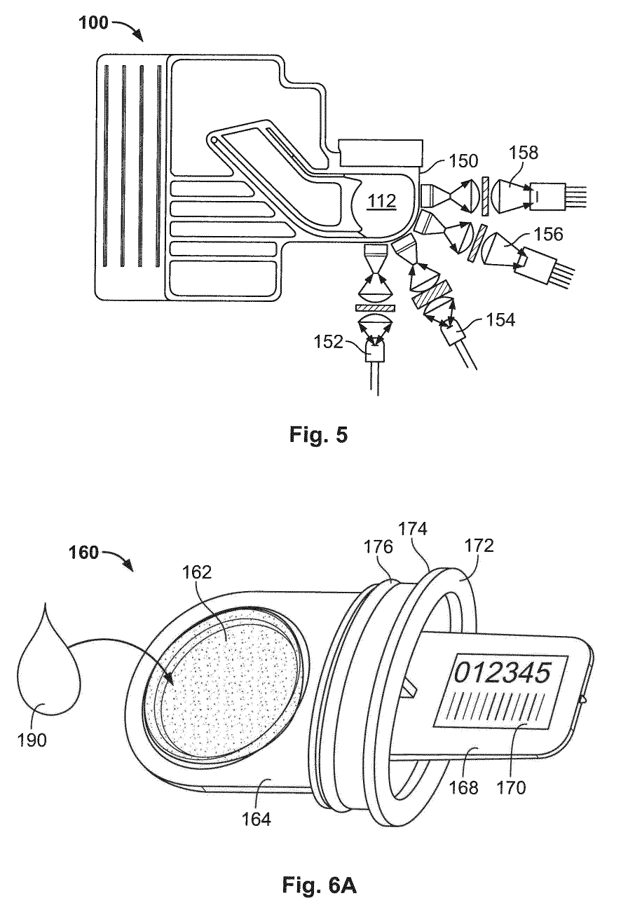

With reference again to FIG. 1B, the reaction card further comprises an optical window 150 adjacent the reaction chamber. In the embodiment illustrated in FIG. 1B, the optical window is positioned on an edge of the reaction card, and is optically transparent to permit periodic or continuous monitoring of reactions that occur in the reaction chamber. For example, for detection of a nucleic acid analyte, periodic or continuous monitoring for amplicons during a polymerase chain reaction can be performed using an optical detection instrument which observes the reaction chamber via the polished optical window. This is depicted in FIG. 5, where reaction chamber 112 of reaction card 100 has an optical window 150. During use of the card in an assay process, the optical window is positioned adjacent a series of light emitting diodes (LEDs), such as a blue LED 152 and a red LED 154. One or more photosensors, such as a green photosensor 156 and a magenta photosensor 158, detect emissions from the reaction chamber that correlate with the presence or absence of an analyte. For example, the magenta photosensor detects Quasar 670 fluorescence while the green photo sensor detects FAM fluorescence when excited with the red and blue LEDs, respectively. A skilled artisan will understand that the necessary connections and electrical interfaces with the LEDs and photosensors are provided in an instrument which the reaction card is inserted for use. The optical window is polished or finished to enable the highest optical clarity feasible for continuous optical transmission. The wail thickness is minimized without compromising the integrity and structure of the reaction card. Furthermore, the inside and outside walls along the window are preferably parallel to one another to minimize optical distortions. In another embodiment, optical lenses are molded on the inside wall of the optical window to support and enhance the external optical detection instrument. Here, the geometry is a smooth curvature, but can be other geometries, such as straight lines resulting in a right-angle. In another embodiment, the optical window is a multi-facet optical window comprised of multiple straight lines.

### B. SAMPLE INTRODUCTION MODULE (SIM)

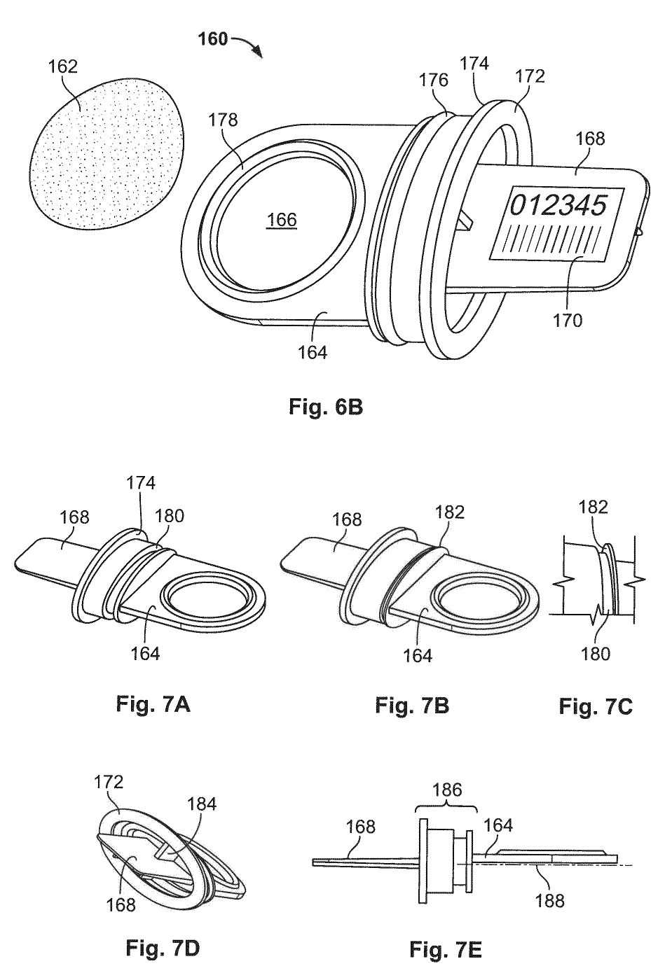

As described above, the reaction card includes a port for receipt of a sample, and in a preferred embodiment, for receipt of a sample carried on a sample introduction module (SIM). An embodiment of a SIM is shown in FIGS. 6A-6B, where a SIM 160 is comprised of a carrier 162 sized to fit within a holder 164. In one embodiment, the holder is formed to include a recessed area 166 into which the carrier is positioned. The SIM also includes a handle 168 for holding the SIM by a user. The handle can include an optional identifier tag 170 with information to identify the patient, sample, date, etc., which can be read by a human or a machine, as in the form of a bar code or RFID.

The SIM is dimensioned for insertion into the port on the reaction card, described above. In this embodiment, the SIM includes a lip 172 having an inner edge 174 that contacts the flange (such as flange 134 on port 104, seen best in FIG. 3A), and in particular contacts the outer surface of the flange on the reaction card. SIM also includes, in one embodiment, a sealing means 176. The sealing means can be an O-ring, sealant, polymer, etc. that ensures a snug fit of the SIM into the port, and preferably a liquid tight fit of the SIM into the port.

The view of the SIM in FIG. 6B, wherein the sample carrier is shown removed from the holder, permits a view of the features in holder 164 that permit affixing the carrier in the holder, and specifically within recessed area 166. Here, edge 178 can include conformational or material features that act as energy directors to focus and facilitate affixing the carrier to the holder, for example by ultrasonic or laser welding. Preferably, the surface area of edge 178 is sufficient to ensure bonding of the carrier to the holder.

FIGS. 7A-7D are various perspective views of a SIM, to show additional features of the SIM in various embodiments. For the convenience of the reader, like structural elements from FIGS. 6A-6B are given like numerical identifiers, even though the sample introduction modules are different embodiments. FIG. 7A is a perspective view of a SIM showing a groove 180 in which a sealing member is disposed. Inner edge 174 that abuts the flange of a reaction card with the SIM is inserted is also visible in FIG. 7A. FIG. 7B shows an embodiment where a wiper 182 is inserted in groove 180. The wiper can serve as a sealing means, and will physically bend upon insertion of the SIM into the port, as can be envisioned from the close up view in FIG. 7C where the wiper does not entirely fill groove 180.

FIG. 7D is a perspective end view of a SIM showing the handle member 168 and lip 172. One or more support ribs, such as rib 184. Rib 184 supports, secures and stabilizes a junction 186, denoted in FIG. 7E, between handle 168 and carrier 164. Junction 186 comprises the sealing member and the lip, and serves as the point of engagement when SIM is inserted into the SIM port on a reaction card.

The geometry of the junction may be circular, elliptical, or other suitable shapes. In one embodiment, the carrier and/or junction is (are) polarized to that the SIM is insertable into the reaction card port in one direction only. Also, as seen best in FIG. 7E, the carrier can be from the centerline of the SIM, the centerline identified by dashed line 188.

With reference again to FIG. 6A, the SIM is intended for receiving a sample 190. The sample, in one embodiment, is treated prior to dispensing on the carrier member. In another embodiment, the sample is a naïve sample prior to application to the carrier, and the carrier can optionally contain one or more reagents for processing or treating the sample. The carrier member can be an absorbent pad or non-absorbent pad, depending on the sample and whether or not it is pretreated prior to dispensing on the carrier of the SIM. The carrier member can also be a glass fiber membrane, a material with a fixed pore size, cellulosic membranes, polypropylene membranes, and membranes made of other polymeric materials. The pore size, pore arrangement and surface chemistry of the carrier are selected in accord with the, and define what, analyte is desired to be captured or retained on the carrier. The geometry of the carrier varies depending on one or more of the (1) type of specimen, (2) amount (e.g. volume) of specimen required, and (3) rate of separation desired. The geometry generally complements the design and geometry of the holder (i.e., it does not need to be restricted to a circular format as shown in FIGS. 6-7).

As mentioned above, the sample can be applied to the carrier as a naïve sample or as a treated sample. As an example, when the sample is blood, the blood can be treated to separate plasma from the cellular components or the blood or the cellular components can be treated with a lysing agent or with an anti-coagulant. In one embodiment, a blood sample is treated with a lysing agent to release nucleic acid from the cells, and the carrier is selected to capture and retain the release nucleic acid upon application of an aliquot of treated blood sample to the carrier. More generally, the sample can be any biological or environmental sample, including but not limited to urine, saliva, plasma, serum, tissue, sputum, mucus, nasal fluid, throat fluid, vaginal fluid, feces, soil, water, plant tissues, and the like. The analyte in the sample can be a nucleic acid (RNA or DNA), proteins, carbohydrates, lipids toxins, including toxins from the subject, from a pathogen (viruses, bacteria, fungi and parasites) infecting the subject, or from the environment. The analyte can be a nucleic acid sequence from the subject or from a viruses, bacteria, fungi, or parasite that has infected the subject. In one embodiment, the analyte is a whole cell, a cell nuclei or other cellular organelle.

In one embodiment, the carrier material on the SIM comprises a component that treats the sample and/or captures an analyte suspected of being in the sample. With regard to a component that treats the sample, the component can be a lysing agent, such as a detergent, or an anti-coagulant, such as heparin or warfarin. With regard to components that can be immobilized on the carrier for capture of a specific analyte of interest, the analyte can be physically entrapped, covalently bonded or non-covalently bonded to the carrier material, by, for example, immuno-complex formation between an immobilized antibody and the analyte of interest. Another example is binding of specific nucleic acids using a complementary nucleic acid strand immobilized on the carrier. The SIM and reaction card can be used in protein assays, immuno-assays, nucleic acid amplification, cell counting assays and assays for carbohydrates and other biological markers. As will be described below, the SIM and reaction card interface with an instrument which is capable of performing the aforementioned assays.

### C. REAGENT RESERVOIRS

The reaction card upon receipt of a sample, generally by insertion of a SIM carrying a sample into the SIM port on the reaction card, is intended to conduct an assay on the sample to detect the presence or absence of an analyte in the sample. In this section, approaches for application or introduction of one or more reagents for conducting the assay are described. When a reaction card includes a reagent, the reaction card and reagent together form what will be referred to as an assay card.

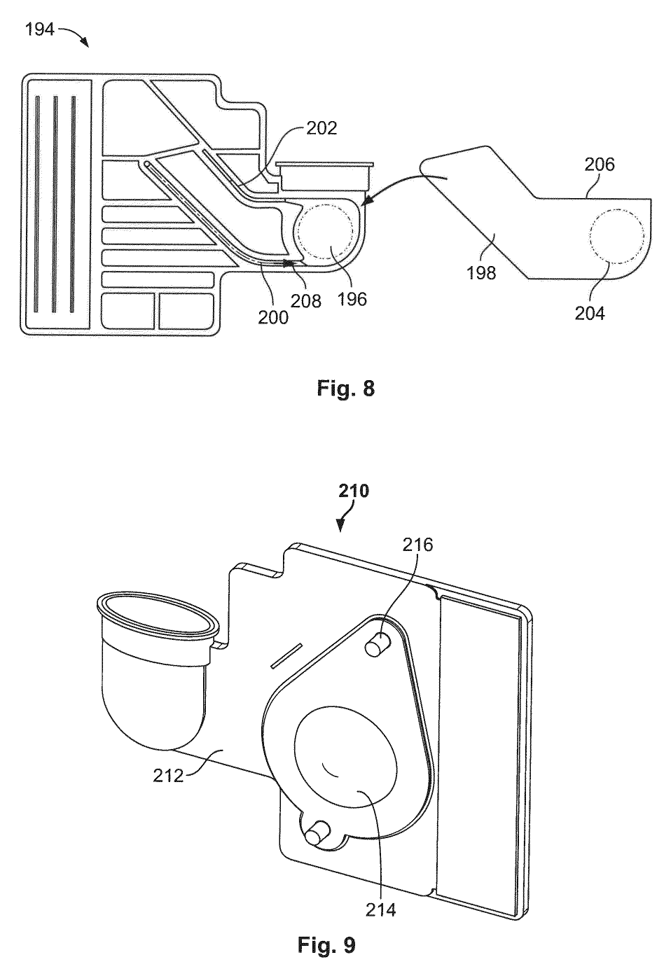

In a first embodiment, one or more reagents can be deposited directly on the reaction card after its manufacture. As shown in FIG. 8, one or more reagents can be disposed on reaction card 192 in one or more locations. For example, a reagent can be deposited in a reaction chamber 196, the deposited reagent in the drawing denoted as being applied in the area enclosed by a dashed circle. A reagent can also be deposited on a wall 198 that encloses the reaction chamber and, in this embodiment, an input channel 200 and an exit channel 202. Reagent on wall 198 is denoted in shadow by the region bounded by dashed line 204, as the reagent deposit will be on an inner facing side 206 of wall 198: that is the side of wall 198 that is facing reaction chamber 196 so that the reagent is available to the reaction. In other embodiments, a reagent is coated or placed in input channel 200. Irrespective of where the reagent is disposed, it can be deposited in the desired location in the form of a freeze dried pellet, lyophilized particulate, gel, or as a dried thin film of reagent material deposited from a solvent. If the reagent is in the format of pellets, the pellets may be deposited in the reaction chamber or in the reagent input channel, where arrow 208 shows direction of liquid flow into the reaction chamber, if a reagent is in the format of a thin film, the film may be located on one or both walls of the reaction chamber.

In another embodiment, the reagent is provided to the reagent card from an externally attached reagent reservoir. This embodiment will be described now with reference to FIGS. 9-15.

FIG. 9 is a schematic of an assay card 210 comprised of a reaction card 212 and a reagent reservoir 214. Reagent reservoir 214 is secured by means to be described to the back face the reaction card, the back face having one or more posts, such as post 216, to guide positioning of the reagent reservoir into its correct alignment, to be described, on the reaction card.

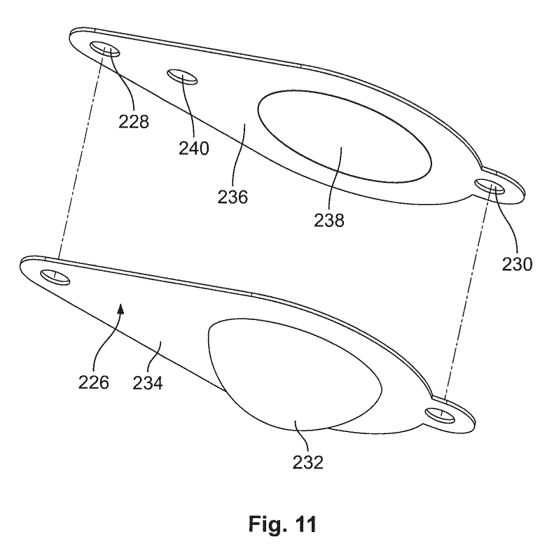

Positioning of the reagent reservoir on the reaction card is illustrated in FIGS. 10A-10B and FIG. 11. In FIG. 10A, the back face of reaction card 212 is shown, with alignment posts 216, 218 indicated. A reagent input port 220 is in fluid communication with an input channel (not visible) in the reaction card, the reagent channel in fluid communication with a reaction chamber, in the area denoted 222 in FIG. 10A where a sample is introduced. An exit port 224 is in fluid communication with an exit channel (not visible in this view) in the reaction card, the exit channel in fluid communication with the reaction chamber. A reagent reservoir 226 is affixed to reaction card 212, by positioning a first opening 228 and a second opening 230 over alignment posts 216, 218, as shown in FIG. 10B. FIG. 11 shows reagent reservoir 226 with a storage chamber 232 for holding a reagent. The storage chamber is surrounded by an elongate collar 234, with openings 228 and 230 in the elongate collar. The storage chamber is constructed to be frangible, so that it bursts open upon application of force. Reagent reservoir 226 is affixed to the reaction card by an adhesive member 236 configured to mate with the reagent reservoir. Adhesive member 236 has a central opening 238 through which reagent in the storage chamber flows upon its release. Adhesive member also includes an opening 240 positioned to align with reagent input port 220 when the reagent reservoir is secured to the reaction card. In one embodiment, the adhesive member is a double-sided adhesive member.

In some embodiments, such as that exemplified in FIG. 11, the reagent reservoir is comprised of two separate foil laminate sheets, one of which is cold formed into a blister or storage chamber (232), while the second foil laminate forms the lidstock or elongate collar (234). Liquid reagent required for an assay is stored in the blister and heat sealed by the lidstock; creating a vapor; oxygen, and ultraviolet (UV) seal. The sealed blister (1) enables storage of the reagent reservoir at ambient temperatures; (2) removes the necessity of cold chain technology; (3) makes it amenable for point-of-care diagnostics in resource-limited settings. By applying controlled force on the frangible/burstable storage chamber (also referred to as a blister pack), the chamber peels or bursts open in a desired direction and dispenses the stored liquid reagent. Exemplary reagent reservoirs with frangible storage chambers are described, for example, in WO 2010/091246, which is incorporated by reference herein in its entirety.

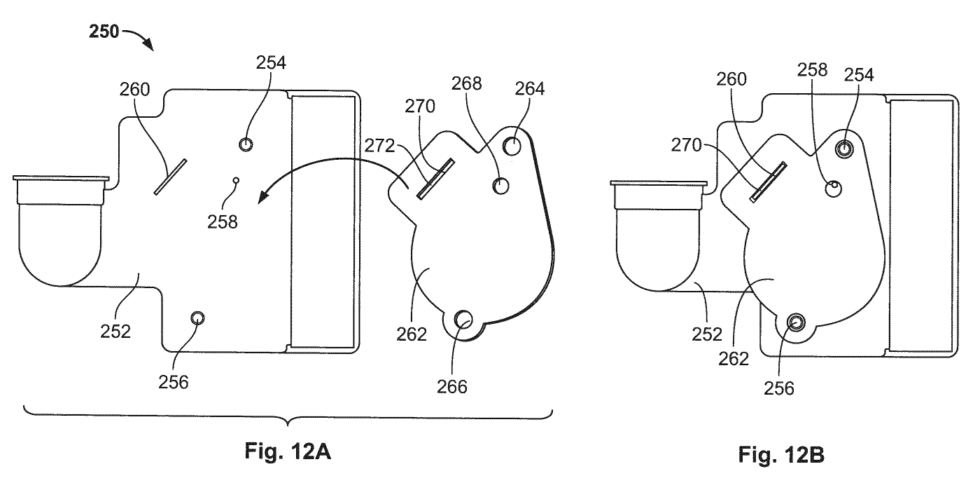

Another embodiment for affixing a reagent reservoir to an external surface of a reaction card is shown in FIGS. 12A-12B. A planar reaction card 250 is comprised of a planar base 252 comprising on one surface one or more alignment posts, such as posts 254, 256, a reagent input port 258 and an exit slot 260. An attachment member 262 comprises first and second openings 264, 266 through which posts 254, 256 insert, as seen in FIG. 12B. An opening 268 in the attachment member aligns with input port 258. At least a portion of the attachment member comprises a liquid impermeable, gas permeable membrane, and in the embodiment shown in FIGS. 12A-12B, a liquid impermeable, gas permeable membrane 270 is affixed to or within a slit 272 that aligns with exit slit 260 on the reaction card. The membrane can be, for example, a polypropylene or silicone film, both of which are impermeable to liquids yet permeable to air. The membrane is preferably capable of withstanding moderate liquid pressure build-up from within the reaction card.

Preferably, attachment member is a double-sided adhesive that bonds to a reagent reservoir on one side and to the reaction card on the other. Preferably the adhesive is an assay-compatible transfer adhesive that bonds well to low surface energy plastics. As can be appreciated, the profile of the attachment member is configured to match with a reagent reservoir. The attachment member may be die cut or laser cut to the desired geometry, of which FIGS. 12A-12B are merely exemplary. A release liner on each side of the attachment member, first and second release liners (not shown in FIGS. 12A-12B) are removed sequentially to affix the attachment member to the correct position on the reaction card. Uniform pressure is applied to the transfer adhesive to ensure a secure bond to the reaction card. The second release liner is removed in preparation for bonding a reagent reservoir and/or liquid impermeable, gas permeable membrane to the outward facing surface of the attachment member.

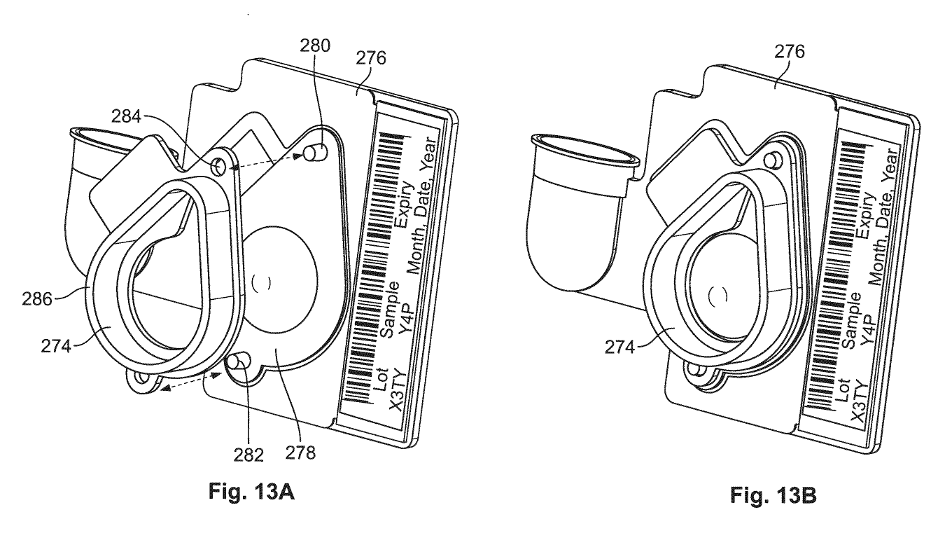

In another embodiment, an optional clamp is affixed to the reagent reservoir secured to a reaction card. Exemplary clamps will be described with respect to FIGS. 13-15, FIGS. 13A-13B show a clamp 274 as it is being positioned (FIG. 13A) and in position (FIG. 13B) on a reaction card 276. A reagent reservoir 278 is in position on the reaction card, where openings in the reagent reservoir are inserted over alignment posts 280, 282. First and second openings in clamp 274, such as opening 284, are positioned to ensure proper alignment of the clamp on the reaction card. The clamp comprises a wall 286 that encloses at least the storage chamber on the reagent reservoir, to protect the chamber during storage and shipping.

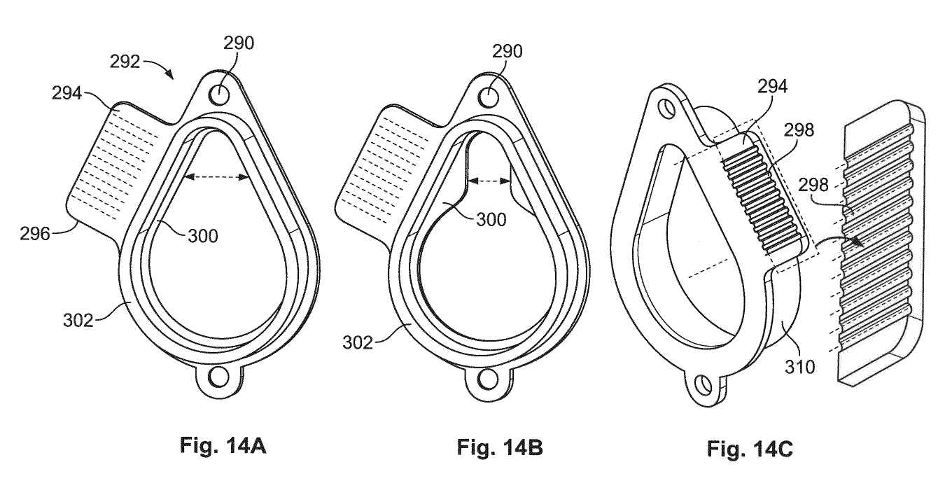

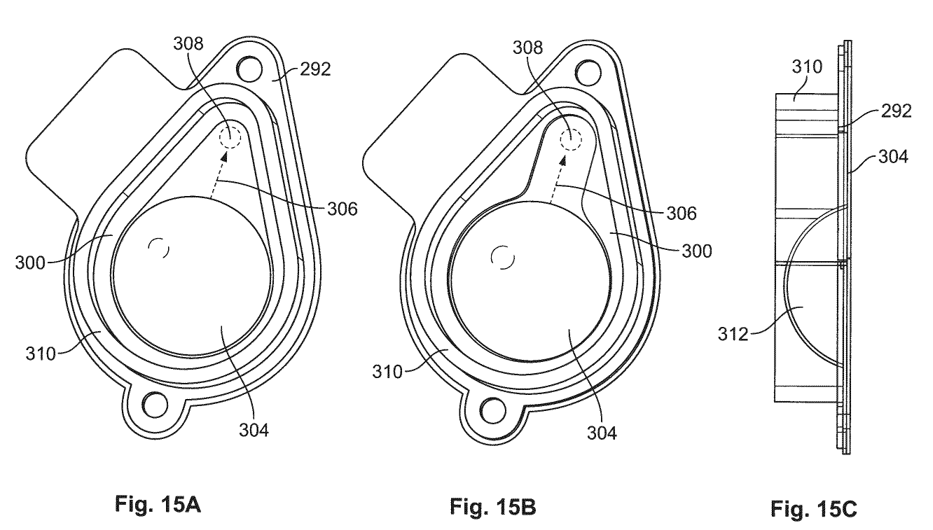

FIGS. 14A-14B are illustrations of various embodiments of clamps. In all embodiments, the clamp geometry is designed to complement the geometry of the reagent reservoir and the reaction card, and a skilled artisan will appreciate that the embodiments shown are merely exemplary of the possible geometries. The clamps typically include at least one alignment means, such as an alignment hole 290 on clamp 292 of FIGS. 14A. The alignment hole complements the position of an alignment post on the reaction card and allows a user to align the clamp easily with the reaction card and its other components, such as the attachment member and reagent reservoir. In some embodiments, a tab 294 provides structural support to a liquid impermeable membrane on the reagent reservoir and prevents the membrane from ballooning during liquid filling. An underside 296 of the tab, the tab side which makes contact with the liquid impermeable membrane, can be textured with ridges and grooves 298, as shown in FIG. 140. This allows for air ventilation even if the extended tab is intimately pressing up against the membrane. The textured geometry may be altered to other textures that would also facilitate air ventilation. In some embodiments, a flange 300 is configured to accommodate the size of a reagent reservoir and to direct the reagent reservoir peeling in a unidirectional pathway (i.e., towards the liquid input port on the reaction card). The clamps shown in FIGS. 14A-14B differ in the geometry of the flange, where the neck region of the flange in FIG. 14B is narrower than that in FIGS. 14A.

FIGS. 15A-15B show the clamps of FIGS. 14A-14B positioned over a reagent reservoir 304. Preferably, there is minimal gap between the flange 300 and edge of the reagent reservoir to minimize the dead volume left behind in reagent reservoir after release of its contents. The narrower neck geometry of the flange in FIG. 15B reduces the dead volume, but may increase the force required to peel the reagent reservoir heat seal. The storage chamber or burstable blister pack on the reagent reservoir when forced open releases its contents, and the contents flow in the direction indicated by arrows 306 toward the input port 308, shown in shadow in FIGS. 15A-15B. The clamp has a collar 310, which is most visible in the perspective drawing in FIG. 140, but also indicated in FIGS. 15A-15C. The height of the collar, e.g, the degree to which the collar extends from the base of the clamp, is preferably greater than the height of the storage chamber on the reagent reservoir 304, so that the collar protects the reagent reservoir during transportation and manual handling (i.e., prevention of accidental pressure being applied). This feature is best seen in FIG. 150, which is a side view of clamp 292 positioned over a reagent reservoir, and collar 310 has a height greater than the storage chamber 312 of the reagent reservoir integrated with a reagent reservoir. The taller collar height protects the reagent reservoir during packaging, shipping, and storage.

The clamp may be fabricated from most any plastic or polymer capable of supporting moderate forces (generally less than about 15 lb-f, or less than about 20 lb-f) without breaking under stress, in some embodiments, the clamp is injection molded from polypropylene such that it can be heat-staked to the alignment posts, which are also molded from polypropylene, on the reaction card for final packaging.

The reagents or reagent stored in the reagent reservoir or applied as a coating to or deposited within the reaction card are readily known to those of skill in the art according to the desired assay to be performed. Typically, the reagent composition in a storage chamber of a reagent reservoir will be a liquid, and the reagent composition when applied or deposited within the reaction card's input channel or reaction chamber will be in dried form, as a pellet or lyophilized particles. The reagent composition, whether liquid or dried, may include a PCR mix comprising a thermo-stable DNA polymerase that could be a hot-start enzyme, dNTPs, forward and reverse primers and/or labeled primers such as Plexor™ primers, fluorescently labeled hybridization oligonucleotides, Molecular Beacons, TagMan probes, Scorpion probes and other probes used for real-time PCR, an intercalating dye such as SYBR™ green or SYTO9, plasmid DNA or other forms of template DNA/RNA, salts such as Tricine, Bicine, (NH₄)₂SO₄, MgCl₂ and MnCl₂, stabilizing sugars such as sucrose or trehalose, other additives such as BSA, gelatin and betaine, etc. The reagent composition may comprise reagents for performing amplification assays such as FOR, Ligase Chain Reaction (LCR), Loop mediated Isothermal amplification (LAMP), Transcription mediated amplification (TMA), Nucleic Acid Sequence Based Amplification (NASBA) and Helicase dependent amplification (HDA). Furthermore the reagent composition may be composed of reagents for performing tests for other biological molecules such as sugars, proteins and lipids using assays such as immuno-assays or other enzyme-based assays. Furthermore, the reagent composition may be composed of reagents for cell based assays such as luciferase assays and pico-green based assays.

As can be appreciated from the foregoing, another aspect of the invention is a kit for use in determining the presence or absence of an analyte in a sample. The kit includes a planar reaction card, as described above. In one embodiment, the reaction card comprises a sample port for receiving a sample into a reaction chamber; a reagent input port on the card for receiving a reagent, the reagent input port in fluid communication with the reaction chamber, and an optical window positioned on the card for monitoring a reaction between the sample and the reagent in the reaction chamber. The kit also includes one or more reagent reservoirs configured to be affixed to the reaction card for release of a liquid component contained in a storage chamber on each of the one or more reagent reservoirs into the reagent input port of the reaction card. The kit also includes a sample introduction module configured for insertion into the sample port on the reaction card.

in one embodiment of the kit, the one or more reagent reservoirs comprise a first reagent reservoir comprising a buffer and a second reagent reservoir comprising reagents for a polymerase chain reaction. In another embodiment, the kit also includes an attachment member for securing a reagent reservoir in the one or more reagent reservoirs to the reaction card. In still another embodiment, the sample introduction module comprises a carrier member comprising a lysing agent, such as a detergent. The carrier member can also include an anti-coagulant.

### D. METHOD OF USING THE REACTION CARD, SAMPLE INTRODUCTION MODULE AND REAGENT RESERVOIR TO PERFORM AN ASSAY

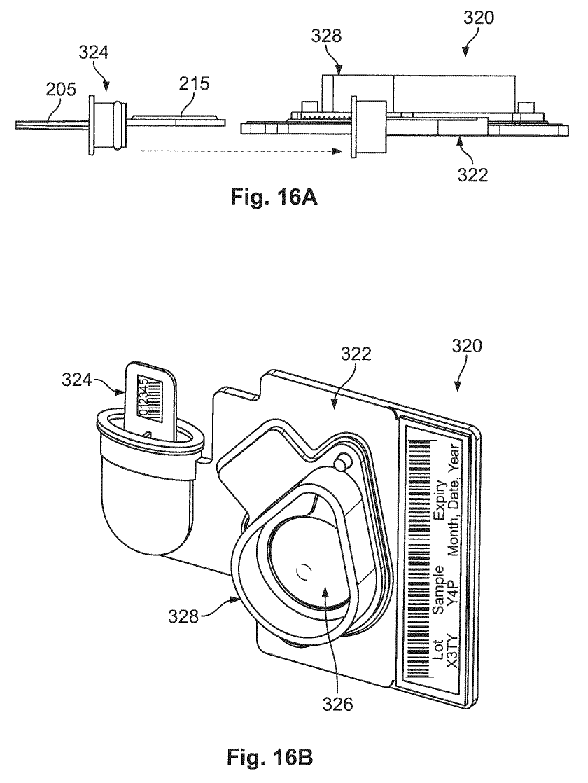

Based on the foregoing, an aspect of the invention is an assay card comprised of a reaction card, a SIM, and a reagent reservoir. Such an assay card 320 is shown in FIGS. 16A-16B, in side view and perspective view, respectively. Assay card 320 is comprised of a reaction card 322, a SIM 324, and a reagent 326. In this embodiment, reagent 326 is included in the assay card as an externally attached reagent reservoir, however a skilled artisan will understand from the discussion in Section C above that the reagent could be deposited within the reaction card. The reagent reservoir is secured to the back face of the reaction card, and protected by a clamp 328.

Methods of using the assay card will now be described, and are merely exemplary of the various processes and procedures which will be understood to vary according to the sample and the analyte of interest. Use of the assay card is described below using assay cards of a geometry different from that described above, merely to illustrate that the overall geometry of the assay card and its components can vary yet retain functionality.

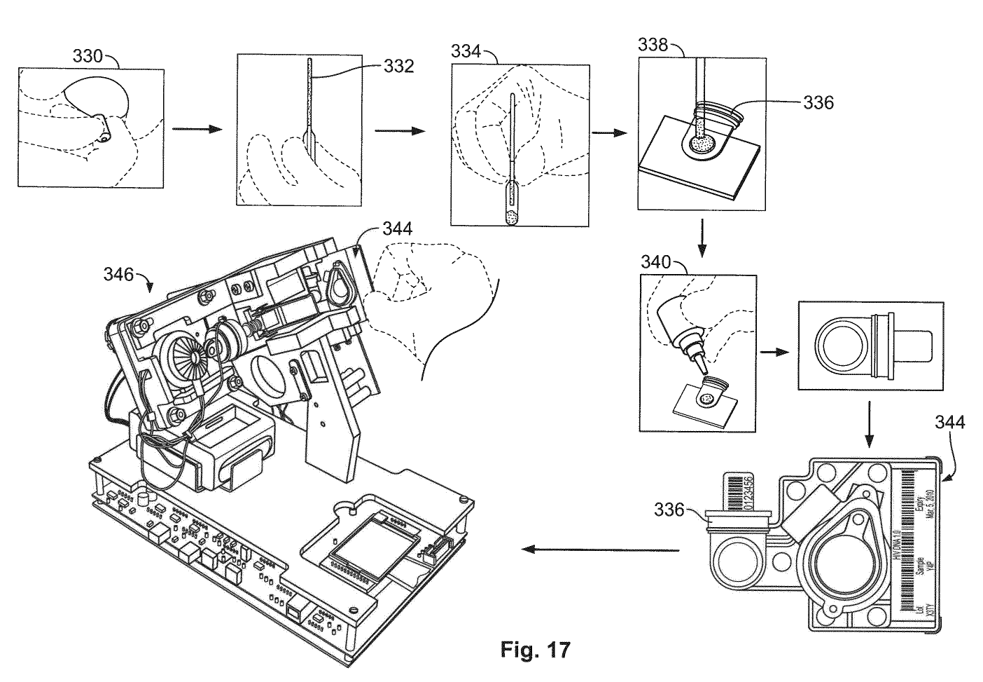

An illustrative embodiment is shown in FIG. 17, where in step 300 blood is drawn, by puncturing a skin site such as a heel of an infant. The blood drop that forms at the puncture site is collected into a collection device 332 containing a lysing reagent, such as a detergent like Triton®-X-100. The blood sample is admixed with the lysing reagent in step 334 and then dispensed onto a carrier in a SIM 336, as shown in step 338. SIM 336 contains a filter membrane such as a glass fiber membrane that traps nucleic acid in the treated sample and separates it from other components of blood that are potent PCR inhibitors via capillary action. A single wash step 340 (e.g. 10 mM NaOH) washes away PCR inhibitors, and the nucleic acid entrapped on the SIM is then introduced into the assay card 342. A label can be affixed to the SIM if desired to identify the sample and other information. The assay card is then placed into an instrument 344 for analysis of the sample, to detect the presence or absence of an analyte. In this example, real-time PCR is conducted by instrument 346.

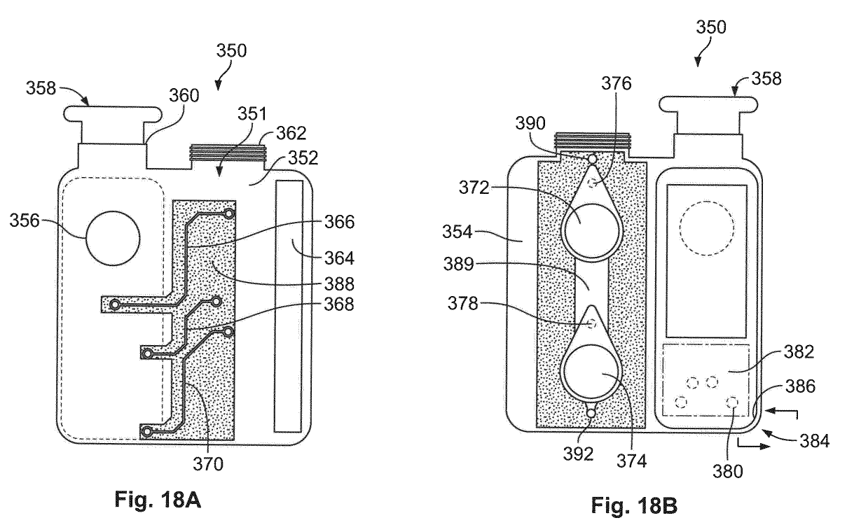

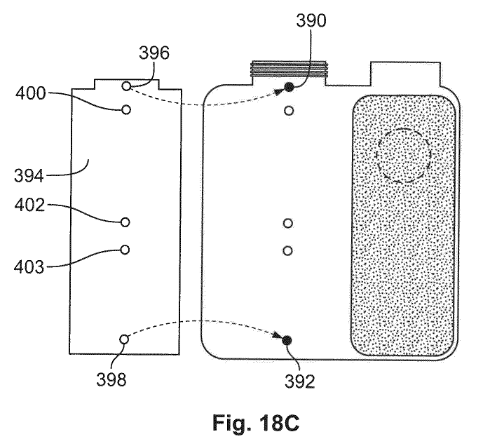

Another example is provided, with reference to FIGS. 18A-18B. An assay card 350 is shown in top view, which includes a reaction card 351 having a first side or front side 362 shown in FIG. 18A and a second or back side 354 shown in FIG. 18B. The front side includes a removable sample cap 356 covering an opening (not visible) in the assay card, the opening providing access to the carrier on a sample introduction module 358. SIM entry port 360 is configured to receive the SIM. The assay card has on one edge a ribbed handle 362 for a user to hold the card. An optional label area 364 is provided to affix assay or patient information.

The front side of the assay card includes at least one channel, and in the embodiment shown, the assay card includes three channels, 366, 368, 370. Two of the channels are for liquid reagents packaged in reagent reservoirs 372, 374, secured to the opposing side (back side 354) of the assay card. The third channel serves to vent the air from the reaction card during input of the liquid reagent. The two liquid channels guide the liquid from the reagent reservoirs into the appropriate chambers of the reaction card. The width, depth, and smooth contour geometry of the reagent input channels are designed such that they enable smooth laminar flow of the liquid reagent and prevent bubble entrapment. There are two reagent entry ports, 376, 378 on the back side of the reaction card which align with the reagent reservoirs. Reagent reservoir 372 contains liquid for washing the sample embedded on the SIM and reservoir 378 contains liquid for reacting with the sample and optionally any solid gel reagents 380 in reaction chamber 382. The reagents can also be stored in the reagent input channel or coated as a film on a wall of the reaction chamber, as described above. The reaction chamber has an optical window 384 at an edge 386 of the assay card. The optical window permits monitoring of the reaction chamber.

Depending on how the reaction card is manufacture, the channels are, in one embodiment, subsequently covered by a thin plastic film 388, e.g., polypropylene, via one of many welding methods, including but not limited to heat sealing and laser/ultrasonic/RF welding. The thin plastic serves to seal the channels and ports where necessary.

With reference to FIG. 18B, a clamp 389 secures the reagent reservoirs to the back side of the reaction card. The clamp is designed to complement the geometry of the reagent reservoirs and reaction card. It is positioned on top of both reagent reservoirs and an optional liquid impermeable, gas permeable membrane affixed over an exit port (not shown) on the back side of the reaction card. Aligning means, such as pins 390, 392, can be provided to assist aligning the reagent reservoirs and the clamps in their proper positions on the reaction card. The clamp may be fabricated from most any plastic (disposable) polymer capable of supporting moderate forces (generally less than about 20 lb-f) without breaking under stress. As shown in FIG. 180, where the card of FIG. 18B is shown with the clamps, reagent reservoirs removed, and an attachment member 394 that secures the reagent reservoirs to the reaction card to the left of the reaction card, the attachment member 394 has openings positioned for alignment with components on the reaction card. For example, openings 396, 398 in the attachment member are positioned for insertion over alignment pins 390, 392, respectively. Attachment member 394 contains holes 400, 402, 404 for alignment with access ports in the reaction card to the liquid channels and exit channel.

In use, a sample is introduced into the assay card by opening the cap 356 and placing a drop of sample on the SIM when it is inserted into the SIM port, as shown in FIG. 18A. The operator can place the cap back in position on the front side of the assay card. The cap creates a vapor and liquid seal with the assay card, which could be realized by one of many methods, including o-ring or friction fit. A snap-lock feature may also be integrated into the design of the cap which would (1) give the operator an audible and tactile feedback that the cap is securely closed, and (2) prevent anyone from re-opening the cap. The cap can also include a flange at the top to prevent the operator from pushing it down too far and damaging the SIM and/or specimen carrier.

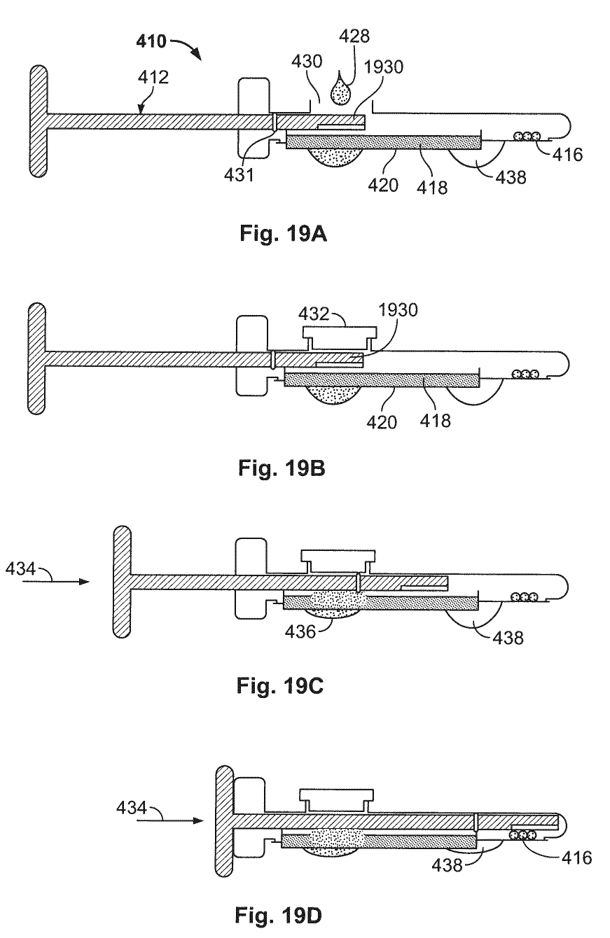

FIGS. 19A-19D present side views of another embodiment of an assay card 410. The assay card comprises a sample introduction member 412 and a reaction card 414, along with reagents 416. The reaction card comprises an absorbent pad 418 positioned on a wall 420. The absorbent pad may fit into the wall by friction fit or be secured using adhesive or epoxy. The height of the wall to hold the absorbent pad is relatively equal to the thickness of the absorbent pad, or slightly less. This allows the pad to have good contact with the sample introduction member. The geometry of the wall and absorbent pad may be most any shape so long as it complements the design of the reaction card and sample introduction member. The absorbent pad has the following specifications: (1) a stiff material that retains is shape even when exposed to liquids, (2) it is manufactured with uniform thickness, (3) its pore size is smaller compared to the specimen carrier, (4) it wicks liquids using capillary action and stores it internally (i.e., it does not spill into other channels or chambers of the assay card), (5) is cut to size such that it can hold the patient sample and wash buffer volume, and (6) it can be die-cut easily.

A procedure for using the assay card 410 to receive, process and analyze a sample, by performing one or more biochemical reactions, includes the following steps. First, a sample 428 is dispensed onto the assay card through a closable opening 430, with care taken to ensure the sample falls directly onto a carrier on the sample introduction member. The sample introduction member engages the reaction card with a liquid tight seal provided by sealing member 431, and is positioned in a first position for receiving a sample. Next, the opening in the assay card is closed using a sample cap 432, as shown in FIG. 19B. The assay card can now be inserted into an analyzer instrument (not shown) for completion of the steps illustrated in FIGS. 19C-19B, The analyzer instrument pushes the SIM in the direction indicated by arrow 434 so that the SIM moves from the sample receiving position to a processing station, and specifically to a first reagent station, for example a wash buffer station. The instrument causes a reagent reservoir 436 containing a buffer to burst open, releasing the buffer from the reagent reservoir and allowing it to flow onto the absorbent pad, as shown in FIG. 190. Next, the analyzer instrument pushes the SIM into a subsequent position, as shown in FIG. 19D, where the sample carrier is positioned with a reaction chamber of the assay card. A second reagent reservoir 438 is burst open by the instrument, to deliver a liquid, such as a buffer, into the reaction chamber, dissolving the freeze/dried gel reagents 416. As the liquid fills the reaction chamber, air vents through the exit channel and liquid impermeable membrane. The biochemical reaction takes place inside the reaction chamber of the assay card, and can be viewed via an optical window at the edge of the card, as described above.

Accordingly, based on the embodiment described with respect to FIGS. 19-A-19D, an aspect of the invention includes an assay card having a sample introduction module movably engaged with an opening on the assay card. When engaged with the assay card the sample introduction module forms a liquid-tight seal with the opening in the assay card. The sample introduction module is movable from a first position to at least one subsequent position in the assay card. One or more reagent reservoirs is/are positioned on the assay card for release of a component when the sample introduction module is in the first position or the at least one subsequent position. In one embodiment, a sample is dispensed onto the sample introduction module when in the first position, the sample dispensed through a sample port in said assay card, the sample port in fluid communication with the sample introduction module when it is in the first position. In another embodiment, the at least one subsequent position is a second position or a third position, and one of the second position and the third position corresponds to a reaction chamber.

In another aspect, methods of using the assay devices and kits described herein are provided. The methods generally comprise providing an assay card or kit as described herein, placing a sample on the assay card, or in one embodiment on the sample introduction module, and conducting a reaction in the reaction chamber between the sample and the reagent. The reaction or absence of reaction between the sample (or an analyte in the sample) is monitored optically via the optical window to detect the presence or absence of an analyte in the sample.

In one embodiment, the sample is a biological sample. In a specific embodiment, the sample is blood, however any of the sample described herein are contemplated for use in the methods. In another embodiment, the analyte is a nucleic acid. In a particular embodiment, the nucleic acid is a viral nucleic acid.

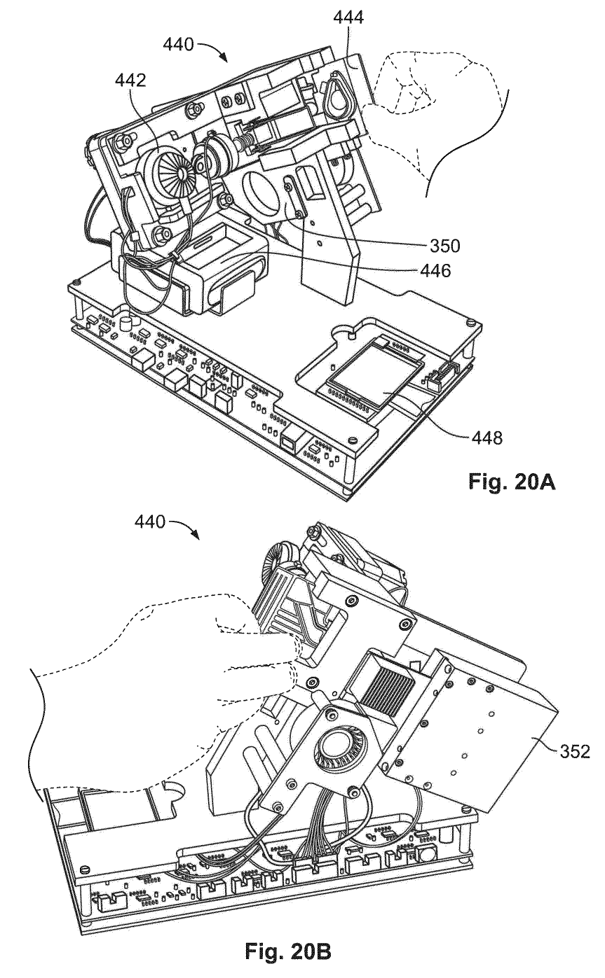

Another aspect of the invention is set forth in the examples, and is directed to a system comprised of an assay card as described above, and an analyzer adapted for receiving the assay card. The analyzer is described with reference to FIGS. 20-22, below in Example 1, and comprises a thermal cycler, an electromechanical component to apply a force to the at least one reagent reservoir to effect release of the liquid reagent, and an optical system.

In one embodiment, the optical system of the analyzer comprises a light source for transmitting light at excitation wavelengths to the reaction chamber and a detector for detecting light at emission wavelengths from the reaction chamber. In another embodiment, the electromechanical component comprises a force sensor. In yet another embodiment, the thermal cycler is in contact with the reaction chamber of the assay card by a thermally-conductive intervening member.

From the foregoing, the features and advantages of the assay card described herein can be appreciated. The assay card permits introduction of a sample, and thereafter does not require pipetting steps by a user to deliver reagents into the reaction card before analysis (e.g. by PCR) of the sample. Manipulation steps required to transfer the sample into the reaction chamber are eliminated. The assay card offers benefits and differs from existing diagnostic test cartridges by, for example: (1) on-board storage of reagents necessary to perform the assay, removing the necessity of any manual pipetting or (reagent) dispensing; (2) one-time use disposable assay card; (3) large surface area to volume ratio in the reaction chamber to facilitate optimal thermal efficiency; and (4) continuous semi-circular polished optical edge for optical (e.g., fluorescence) detection of a reactant; analyte or control. In some embodiments, the assay card interfaces with an instrument capable of real-time PCR. The assay card can also be used for other applications in which other biological specimens are analyzed.

### E. EXAMPLES

The following example is merely for purposes of illustration and is not intended to limit the scope of the subject matter.

### Example 1

### Manufacture of an Assay Card and Use for Analysis of a Sample

### A. Sample Introduction Module (SIM) Fabrication

The SIM membrane holder was an injection molded from Profax-PD702 polypropylene. The DNA capture membrane (Fusion 5™, Whatman Inc. Florham Park, N.J.) is a bound glass fiber membrane that is 11 mm in diameter. The membrane was attached to the membrane holder by ultrasonic welding using a 40 khz Branson Ultrasonic Welder (Model #2000×d/aed=40:2.0, Branson Ultrasonics Corp., Buffalo Grove, Ill.). The membrane holder was placed on top of the membrane and the whole setup was secured to the lower platen of the ultrasonic welder. A cylindrical horn with a diameter of 11 mm was then used to create ultrasonic vibrations at the plastic-membrane interface causing the plastic to melt and bond with the membrane. The welder was setup with the following parameters: Amplitude=100%; Hold time=0.5 s; Pressure=30 psi.

A sample introduction module was prepared as follows. An ultrasonically welded Fusion 5™ membrane was sandwiched between a rectangular piece (1.5″×1″) of parafilm with a 10 mm hole in the center and a 1 inch sq, blotter pad (707, VWR International) such that the membrane and the blotter pad are in good contact. The overhanging flaps of the parafilm were then folded on the back of the blotter pad. The center of the hole on the piece of parafilm was aligned with the ultrasonically welded Fusion 5™ membrane.

### B. Sample Application and Pretreatment

One hundred microliters of blood was spiked with 1000-40,000 8E5 cells that contain a single copy of HIV-1 integrated in their genome. Blood was lysed by mixing with 12.2 μL of 10% Triton™-X-100. The lysed blood was added on top of the membrane in the sample introduction module. The blood lysate wicked into the blotter pad due to capillary action but nucleic acid (DNA) will be trapped on top of the membrane due to its large size. This was followed by addition of 1 mL of 10 mM NaOH to the membrane which washed hemoglobin and other PCR inhibitors leaving the nucleic acid (DNA) entrapped on the membrane carrier of the sample introduction module. The sample introduction module was then ready for insertion into a reaction card (assay card).

### C. Reaction Card Fabrication

The reaction card consisted of a polypropylene film attached to an injection molded base. The base was made of Profax PD-702 polypropylene and the film consists of a polypropylene layer with a coating of ClearFoil® (RPP 37-10280, Roll Print Packaging Inc., Addison, Ill.). The film was laser cut and then cleaned by wiping with isopropanol and is laser welded onto the injection molded base. The injection molded base was cleaned by rinsing in ethanol and Clearweld™ LD-120C (Gentex Corporation, Zeeland, Mich.) infra-red absorbing dye was applied to the area shown in dotted lines in FIGS. 6 and 7 using a Clearweld™ marker. The coated base was placed in a fixture containing a slot which was attached to the lower platen of a Novolas Laser Welder (300 W Line Beam Basic AT Item #B-AT LB300). The laser cut film was overlaid on top of the molded base and a clear polycarbonate clamp was used to hold the film in contact with the molded base. A 45 W laser beam was then swept over the fixture which melted the Clearweld® coated plastic at the interface of the film and base causing the film to join to the base. After laser welding, the reaction card was rinsed in ethanol to remove excess Clearweld®.

### D. Reagent Reservoir Manufacture and Attachment to Reaction Card

A reagent reservoir comprised of two separate foil laminate sheets is prepared, where, one of the foil sheets is cold formed into a blister (26.1124, Roll Print Packaging Inc., Addison, Ill.), and the second foil laminate (RPP 36-1088D, Roll Print Packaging Inc., Addison, Ill.) forms the lidstock. Reagents for a FOR reaction are stored in the blister and heat sealed by the lidstock, creating a vapor, oxygen, and ultraviolet (UV) seal, Briefly, cold formed dimples with diameter=0.58″ and depth=0.2° were made in foil laminate (26-1124) strips (10″×2″) with approximately five dimples per strip. Two holes were punched in the cold formed blister. Rectangular pieces (approx. 2″×2.5″) of the lidstock material were cut and three holes were punched. The cold form blister was then placed on the heat sealing lower platen using the retractable pins for alignment.

The Abbott HIV-1 assay was used for detection of HIV-1 DNA. Approximately 468 μL of PCR reagent (163.8 μl Abbott HIV-1 oligonucleotide reagent, 23.4 μl of 25 mM Manganese Acetate, 18.72 μl Tth DNA Polymerase (3 U/μl) and 262.1 μl nuclease-free H₂O) was pipetted into the blister cavity. For testing blood samples containing 5000 and 1000 HIV-1 copies, the FOR mix also contained 0.2 mg/mL Bovine Serum Albumin (BSA), 150 mM trehalose and 0.2% Tween™-20. For internal control testing on the Quasar 670 channel, HIV negative blood samples were used to prepare the SIM. The PCR mix contained HPR forward primer (100 nM), HPR reverse primer (100 nM), HPR probe (100 nM), dNTPs (0.325 mM), 1.25X RT-PCR buffer (Roche Applied Science), ZO5 (15U), manganese chloride (1.5 mM), and HPR plasmid (30,000 copies), The cavity was then covered with the punched lidstock material with the co-polymer side facing down. The foil laminate and the lidstock were then heat sealed on an in-house designed impulse heat-sealing station (Temperature=220□C and Time=1.2 sec), Alignment holes are punched through both foil laminates which serves to align the two laminates during heat sealing and subsequent attachment to the reaction vessel. A third hole (liquid through port) was punched in the lidstock which serves as the port through which liquid comes out of the blister.

The reagent reservoir was attached to the reaction card using double-sided adhesive (3M 300LSE, 9471 FL). A clamp was heat-staked onto the assay card after the reagent reservoir was attached to direct the peeling of the reservoir on controlled application of force. The peeling of the blister can be directed towards the entry port and channel which would result in the filling of the reaction chamber, An exit channel and exit port has been designed on the injection molded base of the reaction card so that the air can exit the reaction card during blister peeling which otherwise would cause a pressure build-up inside the reaction card preventing reagent entry. The exit port was covered with a 0.45 μm gas permeable liquid impermeable membrane (pin: PP0459025, Sterlitech Corporation, Kent, Wash.) cut into a rectangular piece (3 mm×12 mm) to prevent liquid leakage but to allow air to be vented.

### E. Sample Introduction

Insertion of the sample introduction module carrying the pretreated blood sample (Example 1(B) above) into the assay card makes the card ready for analysis of the sample, by RT-PCR. The sample introduction module entry port of the assay card was layered with double-sided adhesive (3M 300LSE, 9471FL). The adhesive liner was removed prior to sample introduction module insertion. The inserted sample introduction module forms an adhesive seal.

### F. Analyzer Instrument and Sample Analysis

An instrument designed to receive the assay card and conduct the analysis was manufactured. FIGS. 20A-20B present an illustrative embodiment of an analyzer 440, from two different angles. Visible in the angle shown in FIG. 20A is a crusher 442 that bursts open the storage chamber of a reagent reservoir on an assay card 444 inserted into the analyzer. The crusher is further described below as a subsystem of the analyzer, and is also referred to as an electromechanical fluid delivery system. A battery power pack provides portability of the analyzer. A user interface is indicated at 448. Insertion of an assay card into the assay card input slot positions the reaction chamber of the card between two thermal cyclers 350 and adjacent to fluorimeter 352. The analyzer depicted in FIGS. 20A-20B was developed for conducting a polymerase chain reaction on an assay card, and the analyzer comprises the following sub-systems: electrochemical fluid delivery (EFD), thermal cycling and fluorescence detection. The analyzer was driven by a printed circuit board (PCB) developed by Silicon Engines, Arlington Heights, Ill. An LCD display was also added to the system to display the stage of the PCR, block temperature, and ambient temperature.

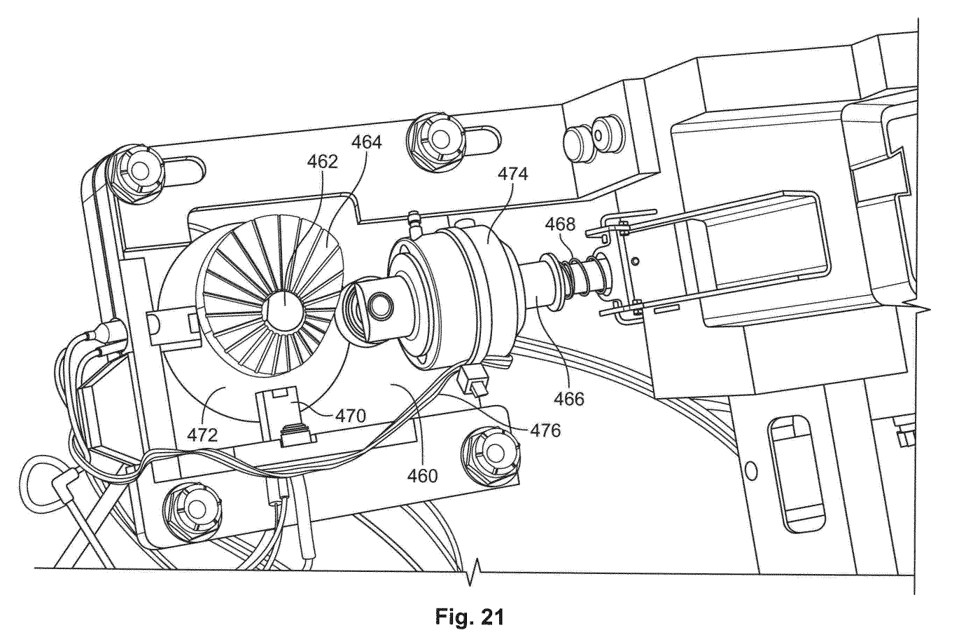

The analyzer includes an electromechanical fluid delivery system. The systems includes a DC motor, a cam, a plunger, two opto-sensors and a strain gauge based force sensor. This subsystem of the analyzer is shown in FIG. 21. In the sub-system shown, relevant components are as indicated: a DC motor 460, and a shaft 462, a cam 464, a plunger 466 with spring 468, two opto-sensors 470, opto-disc 472, a strain gauge based force sensor in housing 474, and clamping lever 476. In one embodiment, the electromechanical fluid delivery system has the capability to deliver up to 440±8 μl to fill up the reaction chamber. A strain-gauge based force sensor is used to detect the bursting event as the storage chamber on a reagent reservoir bursts open. The feedback from the force sensor can be used to determine if the blister pack/storage chamber had the right amount of liquid and if the seals between the reagent reservoir and the assay card were good (not defective). After the bursting event is detected a temporary pause in the system can be implemented to enable all the released air from the blister pack/storage chamber to escape through the gas permeable membrane obstructing the exit port on the assay card. Alternatively, the plunger can be moved forward by a fixed distance without any feedback from the force sensor. In some embodiments the analyzer is driven by a printed circuit board (PCB). In some embodiments, the analyzer includes a user interface/LCD display that shows the stage or cycle of the PCR, the block temperature and the ambient temperature. The LCD can further be used in the field for displaying results, patient ID, assay type and any error flags.

The analyzer also includes an optical sub-system. In one embodiment, the optical system is a fluorimeter (RoMack Fiber Optics, Williamsburg, Va.) having fiber optic bundles, discrete lenses, interference filters, photosensors, LEDs and a printed circuit board (PCB) in an aluminum enclosure was constructed. The fiber optic bundles interface with the optical edge of the assay card on the one side and with discrete lenses on the other. Excitation light from the LEDs is collimated into a beam by a plano-convex lens that passes through an interference filter and is then focused onto the optical fibers. The ends of the fiber optic bundles are attached to plano-convex lenses. The lenses help converge the light from the LEDs into the excitation fibers and converge divergent beams from the emission fibers to obtain a parallel beam that can be refocused onto the photosensor.

The analyzer also includes a thermal cycling subsystem. In one embodiment, a thermal cycler with thermoelectric modules (e.g. Marlow XLT2424) for both heating and cooling was constructed. For selecting an appropriate thermoelectric module, specifications such as ΔT, maximum operating temperature and robustness to temperature cycling were evaluated while surveying thermoelectric modules. The reaction chamber of the assay card interfaces with an aluminum conduction plate which was bonded to the cold surface of the thermoelectric module using graphite adhesive tape (pin: 6838A11, McMaster-Carr). The hot surface of the thermoelectric module was bonded to a heat-sink (pin: 831153B01000), Two centrifugal blower fans were used for dissipating heat from the heat-sink during the cooling cycle, resistive temperature detectors (RTDs) (F3105, Omega Engineering) were bonded using OB-200 epoxy (Omega Engineering) to each of the conduction plates for feed-back control of the plate temperature. A spring (zz2-1, Century Springs Corp., Los Angeles, Calif.) was used to clamp onto the reaction chamber of the assay card. A proportional integral derivative (PID) controller was developed to control the temperature of the conduction plate within ±0.25° C. of the set point especially during the annealing step of PCR.

The analyzer also includes a graphical user interface (GUI), which for the analyzer used in this study was built by Silicon Engines, Arlington Heights, Ill. Prior to analyzer setup, the electromechanical fluid delivery subsystem was adjusted so that when the plunger is in a fully extended position, the gap between the plunger face and the back of the assay card was 1.05 mm. The extend tab in the crusher tab was clicked to actuate the plunger which delivers the PCR reagent mix into the reaction chamber. After complete extension, the plunger was retracted and the assay card removed. The gas permeable membrane was then covered with polyimide tape followed by aluminum foil tape (3M 1450). The assay card was inserted back into the analyzer, the plunger was extended and the PCR cycling protocol was started. In a “detection” tab of the GUI, the gain was set to 1 on both the channels. At every cycle 256 samples were collected at 60 S/s and averaged by the firmware.

The temperature, fluorescence and crusher (electromechanical fluid delivery) log files were obtained from the data storage card on the analyzer and analyzed using MATLAB and Microsoft Excel, The average fluorescence data (N=256) from every cycle was smoothed using a three point average (Sₙ=(Fₙ₋₁+Fₙ+Fₙ₊₁)/3 where n is the cycle number), Fluorescence data obtained in the first 10 cycles was used to establish a base line by linear regression. The equation of the background line was then used to calculate background subtracted data. The background subtracted data for the first 10 cycles was used for threshold determination for Ct calculation. The threshold was set at five standard deviations (SD) above the average data from the first ten cycles.

FIG. 22 shows a typical force curve obtained by the force sensor during the crushing process to burst open the blister pack/storage chamber of the reagent reservoir. During the start of stage 1, the plunger first encounters the reagent reservoir and begins to crush it. As the reagent reservoir is crushed the liquid and air inside the reservoir is compressed and the force encountered by the plunger increases until the point at which the peelable/burstable seal on the reagent reservoir fails (stage 2). During stage 2, there is a drop in force because the air and reagent enter the assay card and fill it up. After the reaction chamber fills with reagent, the reagent then flows into the exit channel in the card, toward the exit port, at which point the reagent contacts the liquid impermeable, gas permeable membrane. At this point, the resistance to further reagent pumping increases, as seen in stage 3. Any more reagent that is pumped in during stage 3, serves to cause the film side of the assay card to bulge out which serves to increase the effective area for heat transfer during thermal cycling contributing to a reduction in run-time. Reagent delivery can also be performed by extending the plunger to a fixed point, which results in consistent volume of reagent delivered. However, data from the force sensor can be used to get a constant amount of film flexing from run-to-run which would ensure similar run-to-run assay performance because the thermal profiles would be consistent.

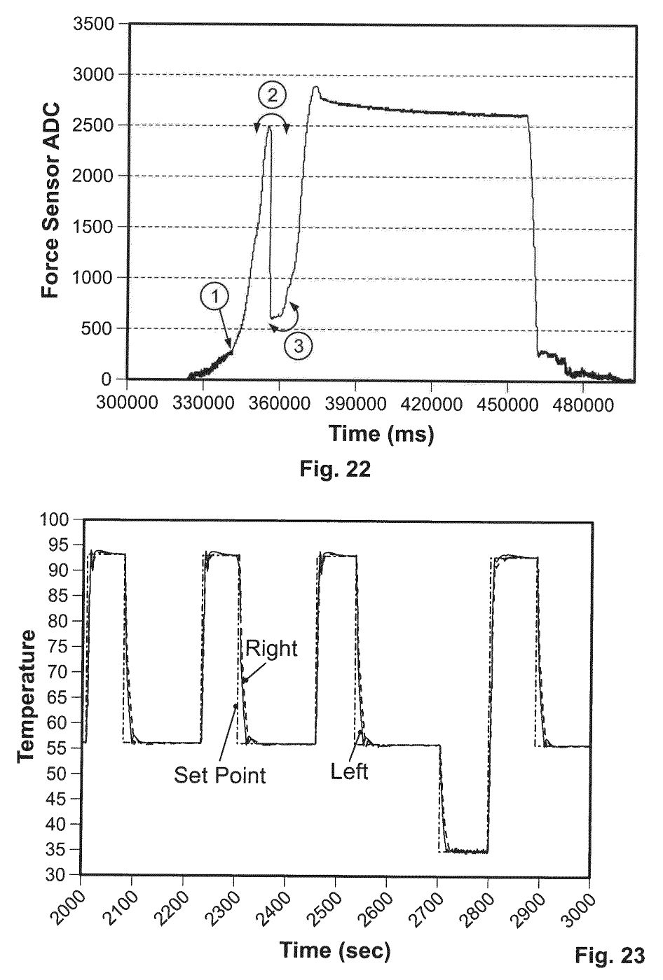

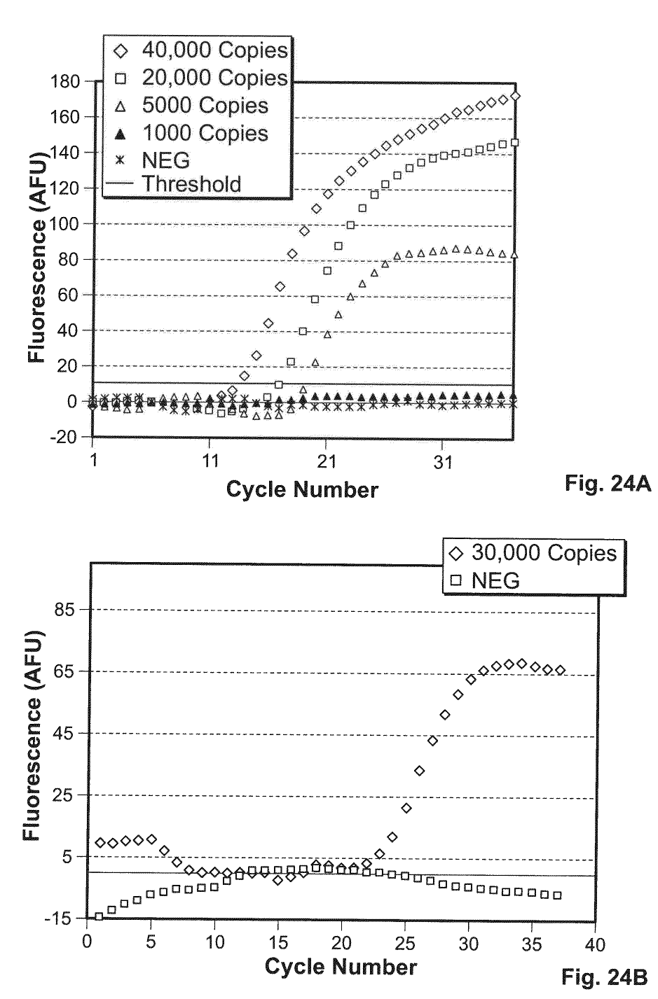

The temperature of the conduction plates were analyzed using MATLAB. It was observed that there were initial peak-to-peak temperature oscillations of 3° C. observed at 95° C. and 93° C. set-points on the left plate which interfaces with the film side wall of the reaction chamber on the assay card. This is shown in FIG. 23, which is a graph of the temperature, in ° C. data on the left and right sleeves of the analyzer as compared to the set point. The oscillations were smaller (±1° C.) on the right sleeve, which interfaces with the molded, rigid side wall of the reaction chamber on the assay card, but the temperature settled within 30 s at the two set points. Similar oscillations were observed at the 56° C. set point. After the temperature settled, it was maintained within ±0.2° C. at the 56° C. and 95° C. set points and within ±0.5° C. at the 93° C. set point on both plates. The plate heating rate was found to be 9.6° C./s and the cooling rate was measured to be 3° C./s. Most commercially available thermal cyclers heat and cool at 2.5-5° C./s and 1.5-2° C./s, respectively. The increased heating rate of the plates helps keep the assay run times similar to those of commercially available cyclers even though the volume of the reagents and sample is larger in the present assay card than it is in most PCR assays. The large surface-to-volume ratio of the reaction chamber of the assay card further facilitates increased heat transfer.

The average fluorescence data (N=256) from every cycle was smoothed using a three point average (Sₙ=(Fₙ₋₁+Fₙ+Fₙ₊₁)/3 where n is the cycle number). Fluorescence data obtained in the first 10 cycles was used to determine the background by linear regression. The equation of the background line was then used to calculate background subtracted data: Bₙ=Sₙ−(m*n+c) where n is the cycle number, m is the slope of the linear regression fit and c is the intercept. The background subtracted data was plotted as shown in FIGS. 24A-24B for FAM and Quasar 670, respectively.

FIG. 24A shows amplification curves obtained with blood samples containing 1000-40,000 HIV-1 copies on the analyzer. The sample with 1000 HIV-1 copies was not detectable. NEG corresponds to the negative control. It was observed that a Ct value≈12 was recorded for a HIV copy number of 40000.

FIG. 24B shows fluorescence data in the Quasar 670 channel after background subtraction. The sample with 30,000 copies was detectable above background while the negative control (NEG) was undetectable. The Quasar 670 channel is used for detecting a fixed amount of internal control plasmid DNA that is present in the PCR reagent mix. The control reports on any PCR inhibition which may result in a failed test. For threshold determination for multiple blood samples with different HIV-1 copy number, the sample showing highest background was used for threshold estimation. Briefly, for all samples the average and the standard deviation of fluorescence data from the first 10 cycles was determined and the threshold determined as follows: Threshold=Average Background+5*SD. The maximum threshold was then used as the global threshold for Ct determination. The Ct values were determined to be 14, 17 and 19 respectively for 40,000, 20,000 and 5000 copies respectively. The sample with 1000 copies was not detected as was the negative control.

The above study shows that the assay card is capable of providing a point-of-care answer regarding the presence or absence of an analyte, as exemplified by HIV-1, wherein RT-PCR was conducted in the reaction chamber. The method using the assay card is simple enough to be implemented as a part of the workflow of rural clinics that perform clinical testing. The small footprint and portability of the analyzer instrument enables testing to be performed in rural clinics with very little bench-space. Furthermore the instrument can be powered by a car-battery enabling the test to be run in mobile testing units. The complete elimination of the PCR reaction assembly step via on-board PCR reagent mix storage simplifies the workflow tremendously.

The illustrations of embodiments described herein are intended to provide a general understanding of the structure of various embodiments, and they are riot intended to serve as a complete description of all the elements and features of devices and systems that might make use of the structures described herein. Figures are also merely representational and may not be drawn to scale. Certain proportions thereof may be exaggerated, while others may be minimized. Accordingly, the specification and drawings are to be regarded in an illustrative rather than a restrictive sense.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

We claim:

1. An assay device, comprising:
a planar reaction card comprising a sample port for receiving a sample into a reaction chamber;
a reagent input port on the card for receiving a reagent, the reagent input port in fluid communication with the reaction chamber,
at least one reagent reservoir in fluid communication with the reagent input port and attached to the card; and
an optical window positioned on the card for monitoring a reaction in said reaction chamber.

2. The device of claim 1, wherein the reaction chamber is directly adjacent the sample port such that upon introduction of a sample via the sample port, the sample is in the reaction chamber.

3. The device of claim 1, wherein the at least one reagent reservoir comprises a storage chamber comprising a reagent in liquid form and an elongate collar enclosing the storage chamber, the elongate collar configured to engage the reaction card to attach the reagent reservoir thereto.

4. The device of claim 3, further comprising an attachment member for securing the reagent reservoir to a face of the reaction card, the attachment member having at least one opening that aligns with the reagent input port when the attachment member is secured to the reaction card.

5. The device of claim 3, further comprising a clamp that encloses the reagent reservoir, the clamp having a collar with a height greater than a height of the storage chamber.

6. The device of claim 1, wherein the at least one reagent reservoir comprises a reagent in dried form deposited in the reaction chamber or in a reagent input channel that connects the reagent input channel and the reaction chamber.

7. The device of claim 6, further comprising a second reagent reservoir attached to an external face of the reaction card by an attachment member, the attachment member having an opening that aligns with the reagent input port on the reaction card.

8. The device of claim 1, further comprising a sample introduction module configured to engage the sample port, the sample introduction module comprising a carrier on which a sample can be captured and a sealing means for engaging the sample introduction module with the sample port.

9. The device of claim 1, further comprising an exit port in fluid communication with the reaction chamber.

10. The device of claim 9, further comprising a liquid impermeable, gas permeable membrane positioned over the exit port.

11. The device of claim 10, wherein the membrane is a part of an attachment member that secures a reagent reservoir to the reaction card.

12. The device of claim 3, wherein the reagent reservoir is comprised of a frangible storage chamber capable of bursting open upon application of force.

13. The device of claim 1, wherein the reaction card has a selected thickness to define an edge with the selected thickness, and the optical window is disposed on the edge in a region adjacent the reaction chamber.

14. An assay card, comprising:
a sample introduction module movably engaged with an opening on the assay card, said sample introduction module forming a liquid-tight seal with the opening when engaged therein;
the sample introduction module movable from a first position to at east one subsequent position in the assay card; and
one or more reagent reservoirs positioned on the assay card for release of a component when the sample introduction module is in the first position or the at least one subsequent position.

15. The assay card of claim 14, wherein a sample is dispensed onto the sample introduction module when in the first position, the sample dispensed through a sample port in said assay card, the sample port in fluid communication with the sample introduction module when it is in the first position.

16. The assay card of claim 14, wherein the at least one subsequent position is a second position or a third position, and where one of the second position and the third position corresponds to a reaction chamber.

17. The assay card of claim 16, wherein the reaction chamber comprises a reagent in dried form.

18. The assay card of claim 16, wherein a reagent reservoir is positioned on the assay card to dispense a liquid reagent from the reagent reservoir through a reagent input port on the assay card that is in fluid communication with the reaction chamber.

19. The assay card of claim 14, wherein the at least one subsequent position comprises a second position and a third position, and a reagent reservoir is associated with the second position to dispense a liquid reagent onto the sample introduction module when positioned in the second position, and a reagent reservoir is associated with the third position to dispense a liquid reagent onto the sample introduction module when positioned in the third position.

20. A kit, comprising:
(a) a planar reaction card comprising;
a sample port for receiving a sample into a reaction chamber;
a reagent input port on the card for receiving a reagent, the reagent input port in fluid communication with the reaction chamber,
an optical window positioned on the card for monitoring a reaction between the sample and the reagent in said reaction chamber;
(b) one or more reagent reservoirs configured to be affixed to the reaction card for release of a liquid component contained in a storage chamber on each of the one or more reagent reservoirs into the reagent input port of the reaction card;
(c) a sample introduction module configured for insertion into the sample port on the reaction card.

21. The kit of claim 20, wherein the one or more reagent reservoirs comprise a first reagent reservoir comprising a buffer and a second reagent reservoir comprising reagents for a polymerase chain reaction.

22. The kit of claim 20, further comprising an attachment member for securing a reagent reservoir in the one or more reagent reservoirs to the reaction card.

23. The kit of claim 20, wherein the sample introduction module comprises a carrier member comprising a lysing agent.

24. A system, comprising:
(a) an assay card, comprising
i) a port for receiving a sample introduction module;
ii) at least one regent reservoir comprising reagent in liquid form, the reagent reservoir positioned on the assay card to release its liquid reagent into a reagent input port on the assay card;
iii) a reaction chamber in fluid communication with the reagent input port, the reaction chamber configured to receive the sample introduction module upon insertion of the sample introduction module into the port; and
iii) an optical window positioned on at least a portion of the reaction chamber for monitoring a reaction in said reaction chamber, and
(b) an analyzer adapted for receiving the assay card, the analyzer comprising a thermal cycler, an electromechanical component to apply a force to the at least one reagent reservoir to effect release of the liquid reagent, and an optical system.

25. The system of claim 24, wherein the optical system comprises a light source for transmitting light at excitation wavelengths to the reaction chamber and a detector for detecting light at emission wavelengths from the reaction chamber.

26. The system of claim 24, wherein the electromechanical component comprises a force sensor.

27. The system of claim 24, wherein the thermal cycler is in contact with the reaction chamber of the assay card by a thermally-conductive intervening member.

28. A method for detecting an analyte in a sample, comprising:
providing an assay card, comprising
a) a sample port for receiving a sample into a reaction chamber;
b) a reagent input port on the card for receiving a reagent, the reagent input port in fluid communication with the reaction chamber,
c) an optical window positioned on the card for monitoring a reaction between the sample and the reagent in said reaction chamber;
d) one or more reagent reservoirs configured to be affixed to the reaction card for release of a liquid reagent contained in a storage chamber on each of the one or more reagent reservoirs into the reagent input port of the reaction card; and
e) a sample introduction module configured for insertion into the sample port on the reaction card;
placing a sample on the assay card;
conducting a reaction in the reaction chamber between the sample and the reagent; and
monitoring the reaction optically via the optical window to detect the presence or absence of an analyte in the sample.

29. The method of claim 28, wherein the sample is a biological sample.

30. The method of claim 29, wherein the sample is blood.

31. The method of claim 28, wherein the analyte is a nucleic acid.

32. The method of claim 31, wherein the nucleic acid is a viral nucleic acid.
